SEQUENCE LISTING (8 sequences)

SEQ ID NO: 1 - DNA, 21 bp, Artificial Sequence
  Synthetic Polynucleotide
agaagaagaaatccgtgtgaa

SEQ ID NO: 2 - DNA, 21 bp, Artificial Sequence
  Synthetic Polynucleotide
gcatccgtggaaatggttaaa

SEQ ID NO: 3 - DNA, 21 bp, Artificial Sequence
  Synthetic Polynucleotide
cattcaggctctggatgaagt

SEQ ID NO: 4 - DNA, 21 bp, Artificial Sequence
  Synthetic Polynucleotide
ggtatcagctctgtgataaca

SEQ ID NO: 5 - DNA, 20 bp, Artificial Sequence
  Synthetic Polynucleotide
cgtattgagacgtaaggcgt

SEQ ID NO: 6 - DNA, 24 bp, Artificial Sequence
  Synthetic Polynucleotide
ctaactcctataacgcgtacgaaa

SEQ ID NO: 7 - DNA, 23 bp, Artificial Sequence
  Synthetic Polynucleotide
gttagaatagtaggtagtttggg

SEQ ID NO: 8 - DNA, 30 bp, Artificial Sequence
  Synthetic Polynucleotide
aaaatacacatataacatttacaaaaactc

(19) United States
(12) Patent Application Publication
(10) Pub. No.: US 2021/0401953 A1
(43) Pub. Date: Dec. 30, 2021

(54) ASPARAGINASE THERAPEUTIC METHODS

(71) Applicants: The Broad Institute, Inc., Cambridge, MA (US); President and Fellows of Harvard College, Cambridge, MA (US); Dana-Farber Cancer Institute, Inc., Boston, MA (US)

(72) Inventors: William Sellers, Brookline, MA (US); Haoxin Li, Cambridge, MA (US)

(73) Assignees: The Broad Institute, Inc., Cambridge, MA (US); President and Fellows of Harvard College, Cambridge, MA (US); Dana-Farber Cancer Institute, Inc., Boston, MA (US)

(21) Appl. No.: 17/293,452

(22) PCT Filed: Nov. 13, 2019
(86) PCT No.: PCT/US2019/061286

§ 371 Date: May 12, 2021

Related U.S. Application Data
(60) Provisional application No. 62/825,665, filed on Mar. 28, 2019.
(60) Provisional application No. 62/760,909, filed on Nov. 13, 2018.

(51) Int. Cl. A61K 38/50; A61K 45/06; A61P 35/00; C12Q 1/6888; G01N 33/574

(52) U.S. Cl. CPC A61K 38/50; A61K 45/06; A61P 35/00; C12Q 1/6888; G01N 2333/982; G01N 33/57446; G01N 33/57438; C12Q 2600/158; C12Q 2600/154; C12Y 305/01001

(57) ABSTRACT

Provided herein, in some embodiments, are methods for detecting a level of asparaginase (ASNS) in a sample obtained from a subject having or at risk for stomach cancer or liver cancer, and methods of treating the subject.

### RELATED APPLICATIONS

This application claims the benefit under 35 U.S.C. § 119(e) of U.S. Provisional Application Ser. No. 62/825,665, filed Mar. 28, 2019, entitled “Asparaginase Therapeutic Methods,” and U.S. Provisional Application Ser. No. 62/760,909, filed Nov. 13, 2018, entitled “Asparaginase Therapeutic Methods,” the entire contents of each of which are incorporated herein by reference.

### FIELD OF THE INVENTION

The present disclosure relates to treatment of gastric and hepatic cancers by administering an effective amount of a pharmaceutical composition comprising asparaginase.

### BACKGROUND OF THE INVENTION

Cancers are diverse in histology, in the pattern of underlying genetic alterations, and in metabolic signatures. Cancer cell metabolic alterations are caused, in part, by genetic or epigenetic changes that perturb the activity of key enzymes or rewire oncogenic pathways. Despite decades of research, understanding cancer metabolic alterations remains elusive, which contributes to the difficulties involved in the identification of predictive metabolic markers and the development of targeted therapeutic strategies.

### SUMMARY OF THE INVENTION

The present disclosure is based, in part, on the finding that asparaginase (ASNS) is differentially present in subpopulations of liver cancers and stomach cancers.

Accordingly, aspects of the disclosure provide methods for treating liver cancer or stomach cancer in a subject comprising detecting a level of asparaginase (ASNS) in a biological sample from a subject, and administering an effective amount of a pharmaceutical composition comprising ASNS to the subject if the biological sample from the subject exhibits a decreased level of ASNS compared to the level of ASNS in a control sample or compared to a predetermined reference level of ASNS.

In some embodiments, detecting a level of ASNS comprises detecting a level of ASNS protein. In some embodiments, the level of ASNS protein is detected by an immunohistochemical assay, an immunoblotting assay, or a flow cytometry assay.

In some embodiments, detecting a level of ASNS comprises detecting a level of a nucleic acid encoding ASNS. In some embodiments, the level of a nucleic acid encoding ASNS is detected by a real-time reverse transcriptase polymerase chain reaction (RT-PCR) assay or a nucleic acid microarray assay.

In some embodiments, detecting a level of ASNS comprises detecting a level of methylation of a ASNS promotor sequence. In some embodiments, the level of methylation is detected using a hybridization assay, a sequencing assay, or a polymerase chain reaction (PCR) assay.

In some embodiments, the biological sample is a tissue sample or a blood sample. In some embodiments, the subject is a human patient having, suspected of having, or at risk for having liver cancer or stomach cancer. In some embodiments, administering ASNS comprises administering ASNS intravenously or intramuscularly.

In some embodiments, the control sample is obtained from a human patient that is undiagnosed with cancer. In some embodiments, the predetermined reference level is a level of ASNS from a human patient that is undiagnosed with cancer.

In another aspect, the present disclosure provides a method for treating liver cancer or stomach cancer in a subject, the method comprising administering to a subject in need thereof an effective amount of a pharmaceutical composition comprising asparaginase (ASNS).

In some embodiments, the pharmaceutical composition is administered to the subject intravenously or intramuscularly. In some embodiments, the pharmaceutical composition comprises ASNS from Erwinia chrysanthemi.

Any of the methods provided herein can further comprise administering to the subject an additional anti-cancer agent.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. The drawings are illustrative only and are not required for enablement of the disclosure. For purposes of clarity, not every component may be labeled in every drawing. In the drawings:

FIG. 1. The Cancer Cell Line Encyclopedia (“CCLE”) database enables quantitative metabolomic modeling in relation to genetic features. (a) 928 cancer cell lines from more than 20 major tissues of origin were profiled for the abundance of 225 metabolites. The number of cell lines is annotated based on the tissues of origin. (b) Schematic summarizing the workflow of metabolite profiling. (c) Heatmap of 225 clustered metabolites (Y axis) and their associations with selected genetic features (X axis). T-statistics were calculated based on linear regression for each metabolite paired with each feature across all cell lines conditioned on the major lineages and were used to represent the regression coefficients scaled by standard deviations. Examples mentioned in the text are magnified and shown outlined by boxes. CN, copy number. (d) 2HG and the top correlated mutations among all mutational features. Cell lines are shown as lines and ordered by increasing levels of 2HG. Those cell lines without corresponding mutations are labeled. The reported test statistics and p-values are based on the significance tests of genetic feature regression coefficients (cell line n=927, two-sided t-tests). (e) Cancer cell lines with outlier levels of 2HG have specific IDH1/2 mutations. (f) Malate levels and a heatmap representation of top correlated copy number alterations among all copy number features. The reported test statistics and p-values are based on the significance tests of genetic feature regression coefficients (cell line n=912, two-sided t-tests). (g), Schematic of the genomic locus containing ME2, ELAC1, and SMAD4.

FIG. 2. Systematic evaluations of metabolite associations with gene methylation patterns. (a) Heatmap of 225 clustered metabolites (Y axis) and their associations with selected gene methylation features (X axis). (b) Oleylcarnitine (an example of long-chain acylcarnitines) and the top correlated features among all methylation features. The reported test statistics and p-values are based on the significance tests of DNA methylation feature regression coefficients (cell line n=811, two-sided t-tests). (c) Scatter plot comparing SLC25A20 DNA methylation levels with its mRNA levels in selected lineages. (d)-(g) Scatter plots comparing SLC25A20 mRNA levels with different acylcarnitines: myristoylcarnitine (d), palmitoylcarnitine (e), stearoylcarnitine (1), and oleycarnitine (g). The q-values were calculated based on the significance test of Pearson correlations (two-sided) with multiple hypothesis testing correction. (h) Scatter plot comparing PYCR1 DNA methylation levels with its mRNA transcripts in hematopoietic cell lines. (i) Scatter plot comparing PYCR1 mRNA transcripts with proline levels in hematopoietic cell lines. (j) Scatter plot comparing GPT2 DNA methylation levels with its mRNA transcripts in hematopoietic cell lines. (k) Scatter plot comparing GPT2 mRNA transcripts with alanine levels in hematopoietic cell lines. For (h)-(k) the p-values were calculated based on the significance test of Pearson correlations (two-sided).

FIG. 3. Systematic evaluations of metabolite-dependency associations. (a) Heatmap of 225 clustered metabolites (Y axis) and their associations with top 3000 gene dependencies (CERES scores) (X axis). The two distinct lipid groups revealed by clustering are highlighted by encircling each group in a dashed line. TAG, triacylglycerol. (b)-(e) T-statistics based on selected metabolites (b) reduced glutathione, (c) oxidized glutathione, (d) NADP⁺, (e) asparagine) and gene dependencies (CERES). Each point represents a gene knockout (KO). The statistical test was based on linear regression conditioned on major lineage types (cell line n=455). (f) Heatmap showing relative levels of ordered TAG species in 928 cell lines. PUFA^{high} and PUFA^{low} cell lines are selected by two-sample t-test (two-sided p<0.05) and are indicated by lines below the heatmap. (g)-(h) Volcano plots comparing the phosphatidylcholine (g) and cholesterol ester (h) species in the PUFA^{high} (n=315) versus PUFA^{low} (n=325) cell lines. Each point represents a metabolite and is colored by the ratio of carbon-carbon double bonds to the acyl chain number. (i) Volcano plot comparing the differential dependencies in the PUFA^{high} (n=315) versus PUFA^{low} (n=325) cell lines. The dependency scores (CERES) used in comparison indicate cell line sensitivity in response to gene knockout (smaller values suggest greater sensitivity). For (g)-(i), the q-values were calculated based on two-sample t-tests (two-sided) with multiple hypothesis testing correction.

FIG. 4. Revealing amino acid metabolism auxotrophs by pooled cancer cell line screens. (a) Scatter plot comparing ASNS DNA methylation levels with ASNS mRNA levels in all cell lines. (b) Schematic summarizing the workflow of pooled cancer cell line screens. (c) Waterfall plots showing the fold changes of pooled CCLE lines (n=554, median of 3 independent cell culture replicates) cultured in RPMI media containing 0.1 μM asparagine, 0.1 μM arginine+1 mM L-citrulline (precursor required for arginine synthesis). For (c), the p-values were calculated based on the significance test of Pearson correlations (two-sided).

FIG. 5. Therapeutic value of asparaginase in stomach and liver cancers. (a) Methylation-specific PCR for ASNS CpG islands (a cropped gel image is shown). This experiment was repeated once. (b) Bisulfite sequencing for ASNS methylation status in different cell lines. Open circles indicate unmethylated CpG while solid circles indicate methylated CpG. This experiment was repeated once with 4 technical replicates for each cell line sample. (c) Cropped immunoblot of ASNS in representative stomach and liver cancer cell lines. Actin was used as the loading control. This experiment was repeated independently twice with similar results. (d) Evaluation of asparagine depletion on the viability of selected stomach and liver cancer cell lines. Viabilities were quantified by Cell-Titer Glo 6 days after treatment (mean±SEM, n=3 cell culture replicates). (e) Volume measurements for tumors resulting from subcutaneous injection of 2313287 cells and SNU719 cells with 3000 units/kg asparaginase treatment or vehicle control (10 tumors from 5 nude mice per condition, mean±SEM). The p-values were calculated based on the tumor volume difference between Day 21 and Day 1 using two-sample t-tests (two-sided). (f) Immunostaining of ASNS in xenograft tumors expressing high (2313287) or low (SNU719) levels of ASNS treated with vehicle control or 3000 units/kg asparaginase 5 times a week for 3 weeks. Each subplot is representative of a different tumor. The immunostaining was repeated independently twice with similar results. Scale bar, 100 μm. (g) Waterfall plots showing the ASNS mRNA levels related to its DNA methylation (probe: cg08114476) in the STAD cohort (n=372) and the LIHC cohort (n=371) in TCGA. Each line represents a tumor sample. The p-values were calculated based on the significance test of Pearson correlations (two-sided).

FIG. 6. Additional information regarding amino acid dependency. (a) Cropped immunoblot of ASNS in A2058 cells with or without dox-inducible ASNS knockdown (KD). Tubulin was used as the loading control. The experiment was repeated independently twice with similar results. (b) Relative cell growth upon ASNS KD with or without rescue in the A2058 cell line grown in DMEM without asparagine (mean±SEM, n=2 cell culture replicates, two-sample t-test, two sided). After 13 days, the relative growth was quantified by standard crystal violet staining. PLK1 KD was used as a control. NEAA, non-essential amino acids. Twelve columns are shown and referred to herein based on their position from left to right. Columns 1, 5, and 9 depict “control.” Columns 2, 6, and 10 depict “ASNS KD1.” Columns 3, 7, and 11 depict “ASNS KD2.” Columns 4, 8, and 12 depict “PLK1 KD1.” (c) ASNS mRNA levels with medians across the CCLE lines grouped by cancer types. DLBCL, diffuse large B-cell lymphoma; CML, chronic myeloid leukemia; AML, acute myeloid leukemia; ALL, acute lymphoblastic leukemia. (d) Scatter plot comparing ATF4 mRNA levels with ASNS mRNA levels in all cell lines. (e) Schematic depicting part of the metabolic pathway of asparagine.

FIG. 7. Evaluation of asparaginase therapeutic value in vivo. (a) Surgically removed SNU719 tumors after asparaginase treatment or vehicle control treatment (2 tumors per nude mouse). (b) Relative mouse body weight changes in the duration of asparaginase treatment (3000 units/kg, 5 times a week) or vehicle control (n=5 nude mice per condition, mean±SEM). Twelve columns are shown and referred to herein based on their position from left to right. Columns 1, 5, and 9 depict control. Columns 2, 6, and 10 depict ASNS KD1. Columns 3, 7, and 11 depict ASNS KD2. Columns 4, 8, and 12 depict PLK1 KD. (c) Methylation-specific PCR for ASNS CpG islands in different tumor samples (a cropped gel image is shown). This experiment was repeated once. (d) Bisulfite sequencing for ASNS methylation status in different tumor samples. Open circles indicate unmethylated CpG while solid circles indicate methylated CpG. This experiment was repeated once with 4 technical replicates for each different tumor sample.

### DETAILED DESCRIPTION OF THE INVENTION

The present disclosure is based, at least in part, on the identification of asparaginase levels, including expression levels and methylation levels, that are differentially present in subpopulations of stomach cancer cells and liver cancer cells. It was determined that subpopulations of stomach cancer cells and liver cancer cells showed lower asparaginase expression levels and higher asparaginase promoter methylation than other cancer cell types.

Thus, some aspects of the present disclosure provide methods for treating stomach cancer or liver cancer comprising detecting the level of asparaginase in a biological sample from a subject, and administering to the subject an asparaginase therapy if the level of asparaginase in the subject's sample is deviated (e.g., decreased) compared to the level in a control sample.

In some embodiments, methods described herein may be used for clinical purposes e.g., for determining the presence of stomach cancer or liver cancer in a sample, identifying patients having stomach cancer or liver cancer, identifying patients suitable for asparaginase treatment, monitoring stomach cancer or liver cancer progression, assessing the efficacy of a treatment against stomach cancer or liver cancer, determining a course of treatment, and/or assessing whether a subject is at risk for a relapse of stomach cancer or liver cancer. The methods described herein may also be useful for non-clinical applications, e.g., for research purposes, including, e.g., studying the mechanism of stomach cancer or liver cancer development and metastasis and/or biological pathways/processes involved in stomach cancer or liver cancer, and developing new therapies for stomach cancer or liver cancer based on such studies.

Methods described herein are based, at least in part, on the discovery that asparaginase is differentially expressed in subpopulations of liver cancers or stomach cancers. Asparaginase that is differentially expressed, in some embodiments, refers to asparaginase that is present at a level in that subpopulation of cells that deviates from a level of asparaginase in a different population of cells. For example, asparaginase that is indicative of stomach cancer or liver cancer may have an elevated level or a reduced level in a sample from a subject (e.g., a sample from a subject who has or is at risk for stomach or liver cancer) relative to the level of asparaginase in a control sample (e.g., a sample from a subject who does not have or is not at risk for stomach cancer or liver cancer). Asparaginase that is indicative of cancer may have a level in a sample obtained from a subject that deviates (e.g., is increased or decreased) when compared to the level of asparaginase in a control sample by at least 10% (e.g., 20%, 30%, 50%, 80%, 100%, 2-fold, 5-fold, 10-fold, 20-fold, 50-fold, 100-fold or more, including all values in between).

Asparaginase is an enzyme that deamidates asparagine to aspartic acid and ammonia. The amino acid sequence of human asparaginase is provided, for example, in UniProt P08243, UniGene Hs.489207, and RefSeq NP_001664.3.

Methods described herein can be used to select a patient for asparaginase therapy. In some embodiments, a patient having a level of asparaginase that is deviated (e.g., increased or decreased) as compared to a level of asparaginase in a control sample is selected for asparaginase therapy. In some embodiments, a patient having a level of asparaginase that is deviated (e.g., increased or decreased) as compared to a predetermined reference level is selected for asparaginase therapy.

### Treatment Methods

A level of asparaginase in a biological sample derived from a subject (e.g., a patient) having or at risk for having stomach cancer and liver cancer can be used for identifying patients that are suitable for asparaginase treatment. Such patients may be identified by comparing the level of asparaginase in a sample obtained from the subject to a level of asparaginase in a control sample or a predetermined reference level.

For example, if the level of asparaginase in a sample from the subject deviates (e.g., is decreased) compared to the level in a control sample or a predetermined reference level, the subject may be identified as suitable for asparaginase treatment. In some embodiments, if a predetermined reference level represents a range of levels of asparaginase in a population of subjects that have stomach cancer or liver cancer, then if the subject has a level of asparaginase that falls within that range, the subject may be identified as suitable for asparaginase treatment.

Methods for treating liver cancer or stomach cancer in a subject, in some embodiments, comprise detecting a level of asparaginase in a sample from a subject and administering an asparaginase therapy to the subject if the level of asparaginase in the sample from the subject is a deviated level compared to the level of asparaginase in a control sample or compared to a predetermined reference level.

As used herein, “a deviated level” means that the level of asparaginase is elevated or reduced as compared to a level of asparaginase in a control sample or as compared to a predetermined reference level of asparaginase. Control levels and predetermined reference levels are described in detail herein, and would be readily determined by one of ordinary skill in the art. A deviated level of asparaginase includes a level of asparaginase that is, for example, 1%, 5%, 10%, 20%, 30%, 40%, 50%, 60%, 70%, 80%, 90%, 100%, 150%, 200%, 300%, 400%, 500% or more deviated from a level of asparaginase in a control sample or a predetermined reference level, including all values in between. In some embodiments, the level of asparaginase in a sample from a subject is at least 1.1, 1.2, 1.3, 1.4, 15, 1.6, 1.7, 1.8, 1.9, 2, 2.5, 3, 3.5, 4, 4.5, 5, 5, 6, 7, 8, 9, 10, 50, 100, 150, 200, 300, 400, 500, 1000, 10000-fold or more deviated from a level of asparaginase in a control sample or a predetermined reference level, including all values in between.

Methods for treating liver cancer or stomach cancer in a subject, in some embodiments, comprises detecting a level of asparaginase in a sample from a subject and administering an asparaginase therapy to the subject if the level of asparaginase in the sample from the subject is decreased compared to the level of asparaginase in a control sample or compared to a predetermined reference level.

As used herein, a “decreased level” means that the level of asparaginase (e.g., level of asparaginase protein) is lower than the level of asparaginase in a control sample or a predetermined reference level of asparaginase. A decreased level of asparaginase includes a level of asparaginase that is, for example, about 1%, 5%, 10%, 20%, 30%, 40%, 50%, 60%, 70%, 80%, 90%, 100%, 150%, 200%, 300%, 400%, 500% or more than about 500% less than a level of asparaginase in a control sample or a predetermined reference level, including all values in between. In some embodiments, the level of asparaginase in a sample from a subject is at least 1.1, 1.2, 1.3, 1.4, 15, 1.6, 1.7, 1.8, 1.9, 2, 2.5, 3, 3.5, 4, 4.5, 5, 5, 6, 7, 8, 9, 10, 50, 100, 150, 200, 300, 400, 500, 1000-fold or more than 1000-fold less than a level of asparaginase in a control sample or a predetermined reference level, including all values in between.

Methods for treating liver cancer or stomach cancer in a subject, in other embodiments, comprise detecting a level of asparaginase promoter methylation in a sample from a subject and administering an asparaginase therapy to the subject if the level of asparaginase promoter methylation in the sample from the subject is increased compared to the level of asparaginase promoter methylation in a control sample or compared to a predetermined reference level.

As used herein, an “increased level” means that the level of asparaginase promoter methylation is higher than a level of asparaginase promoter methylation in a control sample or a predetermined reference level of asparaginase promoter methylation. An elevated level of asparaginase promoter methylation includes a level of asparaginase promoter methylation that is, for example, 1%, 5%, 10%, 20%, 30%, 40%, 50%, 60%, 70%, 80%, 90%, 100%, 150%, 200%, 300%, 400%, 500% or more than 500% increased relative to a level of asparaginase promoter methylation in a control sample or a predetermined reference level. In some embodiments, the level of asparaginase promoter methylation in a sample from a subject is at least 1.1, 1.2, 1.3, 1.4, 15, 1.6, 1.7, 1.8, 1.9, 2, 2.5, 3, 3.5, 4, 4.5, 5, 5, 6, 7, 8, 9, 10, 50, 100, 150, 200, 300, 400, 500, 1000-fold or more than 1000-fold higher than a level of asparaginase promoter methylation in a control sample or a predetermined reference level, including all values in between.

In some embodiments, the subject is a human patient having a symptom of a stomach cancer. For example, the subject may exhibit fatigue, bloating, severe and persistent heartburn, persistent nausea, persistent vomiting, and/or unintentional weight loss, or a combination thereof. In other embodiments, the subject has no symptom of a stomach cancer at the time the sample is collected, has no history of a symptom of a stomach cancer, or has no history of a stomach cancer.

In some embodiments, the subject is a human patient having a symptom of a liver cancer. For example, the subject may exhibit weakness, fatigue, loss of appetite, upper abdominal pain, nausea, vomiting, unintentional weight loss, abdominal swelling, and/or jaundice, or a combination thereof. In other embodiments, the subject has no symptom of a liver cancer at the time the sample is collected, has no history of a symptom of a liver cancer, or has no history of a liver cancer.

Methods described herein also can be applied for evaluation of the efficacy of a asparaginase therapy for a stomach cancer or a liver cancer, such as those described herein, given that the level of asparaginase may be deviated in stomach cancers or liver cancers. For example, multiple biological samples (e.g., tissue samples) can be collected from a subject to whom a treatment is performed, before and after the treatment or during the course of the treatment. The levels of asparaginase can be measured by any of the assays described herein, or any other assays known in the art, and levels of asparaginase can be determined accordingly. For example, in some embodiments, if the level of asparaginase increases after a treatment or over the course of a treatment (e.g., the level of asparaginase in a later collected sample as compared to that in an earlier collected sample), this may indicate that the treatment is effective.

If the subject is identified as not responsive to a treatment, a higher dose and/or frequency of dosage of asparaginase therapy can be administered to the subject. In some embodiments, the dosage or frequency of dosage of the asparaginase therapy is maintained, lowered, increased, or ceased in a subject. Alternatively, a different or supplemental treatment can be applied to a subject who is found not to be responsive to asparaginase therapy.

Also within the scope of the present disclosure are methods of evaluating the severity of a stomach cancer or a liver cancer. For example, as described herein, a stomach cancer or a liver cancer may be in a quiescent state (remission), during which the subject may not experience symptoms of the disease. Stomach cancer or liver cancer relapses are typically recurrent episodes in which the subject may experience a symptom of a stomach cancer or a liver cancer. In some embodiments, the level of asparaginase is indicative of whether the subject will experience, is experiencing, or will soon experience a cancer relapse. In some embodiments, methods involve comparing the level of asparaginase in a sample obtained from a subject having stomach cancer or liver cancer to the level of asparaginase in a sample from the same subject at a different stage or time point, for example a sample obtained from the same subject when in remission or a sample obtained from the same subject during a relapse.

### Asparaginase Therapy

A subject described herein may be treated with any appropriate asparaginase therapy. Examples of asparaginase therapy include, but are not limited to, E. coli asparaginase (ELSPAR®), a pegylated form of E. coli asparaginase (ONCASPAR®), and Erwinia chrysanthemi asparaginase (ERWINASE®).

In some embodiments, asparaginase therapy is administered one or more times to a subject. Asparaginase therapy may be administered along with another therapy as part of a combination therapy for treatment of a stomach cancer or a liver cancer. For example, asparaginase therapy can be administered in combination with chemotherapy. Combination therapy, e.g., asparaginase therapy and chemotherapy, may be provided in multiple different configurations. One therapy may be administered before or after the administration of the other therapy. In some instances, the therapies are administered concurrently, or in close temporal proximity (e.g., there may be a short time interval between the therapies, such as during the same treatment session). In other instances, there may be greater time intervals between the therapies, such as during the same or different treatment sessions.

In some embodiments, a radiation therapy is administered to a subject. Examples of radiation therapy include, but are not limited to, ionizing radiation, gamma-radiation, neutron beam radiotherapy, electron beam radiotherapy, proton therapy, brachytherapy, systemic radioactive isotopes and radiosensitizers.

In some embodiments, a surgical therapy is administered to a subject. Examples of a surgical therapy include, but are not limited to, a lobectomy, a wedge resection, a segmentectomy, and a pneumonectomy.

An immunotherapeutic agent can also be administered to a subject. In some embodiments, the immunotherapeutic agent is a PD-1 inhibitor or a PD-L1 inhibitor. In some embodiments, the immunotherapeutic agent is Nivolumab. In some embodiments, the immunotherapeutic agent is Pembrolizumab.

A chemotherapeutic agent can also be administered to a subject. Examples of chemotherapy include, but are not limited to, platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; topoisomerase I inhibitors, such as Camptothecin, Topotecan, irinotecan/SN38, rubitecan, Belotecan, and other derivatives; topoisomerase II inhibitors, such as Etoposide (VP-16), Daunorubicin, a doxorubicin agent (e.g., doxorubicin, doxorubicin HCl, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; antimetabolites, such as folic family (e.g., Methotrexate, Pemetrexed, Raltitrexed, Aminopterin, and relatives); purine antagonists (e.g., Thioguanine, Fludarabine, Cladribine, 6-Mercaptopurine, Pentostatin, clofarabine and relatives) and pyrimidine antagonists (e.g., Cytarabine, Floxuridine, Azacitidine, Tegafur, Carmofur, Capacitabine, Gemcitabine, hydroxyurea, 5-Fluorouracil (5FU), and relatives); alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives); nitrosoureas (e.g., Carmustine, Lomustine, Semustine, Fotemustine, Nimustine, Ranimustine, Streptozocin, and relatives); triazenes (e.g., Dacarbazine, Altretamine, Temozolomide, and relatives); alkyl sulphonates (e.g., Busulfan, Mannosulfan, Treosulfan, and relatives); Procarbazine; Mitobronitol, and aziridines (e.g., Carboquone, Triaziquone, ThioTEPA, triethylenemalamine, and relatives); antibiotics, such as Hydroxyurea, anthracyclines (e.g., doxorubicin agent, daunorubicin, epirubicin and other derivatives); anthracenediones (e.g., Mitoxantrone and relatives); and the streptomyces family (e.g., Bleomycin, Mitomycin C, Actinomycin, Plicamycin). A subject may also be administered ultraviolet light.

### Non-Clinical Applications

Detection of asparaginase in stomach cancer or liver cancer as described herein may also be applied for non-clinical uses, for example, for research purposes. In some embodiments, the methods described herein may be used to study the behavior of stomach cancer cells or liver cancer cells and/or mechanisms (e.g., the discovery of novel biological pathways or processes involved in stomach cancer or liver cancer development and/or metastasis).

In some embodiments, detection of asparaginase in stomach cancer or liver cancer, as described herein, may be relied on in the development of new therapeutics for a stomach cancer or a liver cancer. For example, a level of asparaginase may be measured in samples obtained from a subject having been administered a new therapy (e.g., in a clinical trial). In some embodiments, a level of asparaginase may indicate the efficacy of a new therapeutic or the progression of cancer in the subject prior to, during, or after the new therapy.

### Analysis of Biological Samples

Any sample that may contain a level of asparaginase can be analyzed by assay methods described herein, or using other assay methods familiar to one of ordinary skill in the art. The methods described herein involve providing a sample obtained from a subject. In some embodiments, the sample may be a cell culture sample for studying cancer cell behavior and/or mechanism. In some embodiments, the sample is a biological sample obtained from a subject. For example, a biological sample obtained from a subject may comprise cells or tissue, e.g., blood, plasma or protein, from a subject. A biological sample can comprise an initial unprocessed sample taken from a subject as well as subsequently processed, e.g., partially purified or preserved forms. Non-limiting examples of biological samples include tissue, blood, plasma, tears, or mucus. In some embodiments, the sample is a body fluid sample such as a serum or plasma sample. In some embodiments, multiple (e.g., at least 2, 3, 4, 5, or more) biological samples may be collected from a subject, over time or at particular time intervals, for example to assess a disease progression or to evaluate the efficacy of a treatment.

A biological sample can be obtained from a subject using any means known in the art. In some embodiments, a sample is obtained from a subject by a surgical procedure (e.g., a laparoscopic surgical procedure). In some embodiments, a sample is obtained from a subject by a biopsy. In some embodiments, a sample is obtained from a subject by needle aspiration.

In some embodiments, a subject has undergone, is undergoing, potentially will undergo, or is a candidate for undergoing, analysis and/or treatment as described herein. In some embodiments, a subject is a human or a non-human mammal. In some embodiments, a subject is suspected of or is at risk for stomach cancer or liver cancer. Such a subject may exhibit one or more symptoms associated with stomach cancer or liver cancer. Alternatively or in addition, such a subject may have one or more risk factors for stomach cancer or liver cancer, for example, an environmental factor associated with stomach cancer (e.g., family history of stomach cancer) or liver cancer (e.g., excessive alcohol consumption).

A subject may be a cancer patient who has been diagnosed as having stomach cancer or liver cancer. Such a subject may be having a relapse, or may have suffered from the disease in the past (e.g., currently relapse-free). In some embodiments, the subject is a human cancer patient who may be on a treatment regimen for a disease, for example, a treatment involving chemotherapy or radiation therapy. In other embodiments, the subject is a human cancer patient who is not on a treatment regimen.

Examples of stomach cancer compatible with aspects of the disclosure include, without limitation, adenocarcinoma, lymphoma, gastrointestinal stromal tumor (GIST), carcinoid tumor, squamous cell carcinoma, small cell carcinoma, and leiomyosarcoma.

Examples of liver cancer compatible with aspects of the disclosure include, without limitation, benign liver tumor, hemangioma, hepatic adenoma, focal nodular hyperplasia, hepatocellular carcinoma (hepatocellular cancer), intrahepatic cholangiocarcinoma (bile duct cancer), angiosarcoma, hemangiosarcoma, hepatoblastoma, and secondary liver cancer (metastatic liver cancer).

Any of the samples described herein can be subject to analysis using assay methods described herein, or other assays known to one of ordinary skill in the art, which involve measuring a level of asparaginase. Levels (e.g., the amount) of asparaginase, or changes in a level of asparaginase, can be assessed using assays known in the art and/or assays described herein.

As used herein, the terms “detecting” or “detection,” or alternatively “measuring” or “measurement,” mean assessing the presence, absence, quantity or amount (which can be an effective amount) of a substance within a sample, including the derivation of qualitative or quantitative concentration levels of such substances.

In some embodiments, a level of asparaginase is assessed or measured by directly detecting asparaginase protein in a sample such as a biological sample. Alternatively or in addition, the level of asparaginase protein can be assessed or measured by indirectly detecting asparaginase protein in a sample such as in a biological sample, for example, by detecting the level of activity of the protein (e.g., in an enzymatic assay).

A level of asparaginase protein may be measured using an immunoassay. Examples of immunoassays include, without limitation, immunoblotting assays (e.g., Western blot), immunohistochemical assays, flow cytometry assays, immunofluorescence assays (IF), enzyme linked immunosorbent assays (ELISAs) (e.g., sandwich ELISAs), radioimmunoassays, electrochemiluminescence-based detection assays, magnetic immunoassays, lateral flow assays, and related techniques. Additional suitable immunoassays for detecting asparaginase protein will be apparent to those of ordinary skill in the art.

Such immunoassays may involve the use of an agent (e.g., an antibody, including monoclonal or polyclonal antibodies) specific to asparaginase. An agent such as an antibody that “specifically binds” to asparaginase is a term well understood in the art, and methods to determine such specific binding are also well known in the art. An antibody is said to exhibit “specific binding” if it reacts or associates more frequently, more rapidly, with greater duration and/or with greater affinity with asparaginase than it does with other proteins. It is also understood that, for example, an antibody that specifically binds to asparaginase may or may not specifically or preferentially bind to another peptide or protein. As such, “specific binding” or “preferential binding” does not necessarily require (although it can include) exclusive binding. An antibody that “specifically binds” to asparaginase may bind to one epitope or multiple epitopes in asparaginase.

As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)2, Fd fragments, Fv fragments, scFv, and domain antibodies (dAb) fragments (de Wildt et al., Eur J Immunol. 1996; 26(3):629-39)) as well as complete antibodies. An antibody can have the structural features of IgA, IgG, IgE, IgD, IgM (as well as subtypes thereof). Antibodies may be from any source, but primate (human or non-human primate) and primatized or humanized are preferred in some embodiments.

Antibodies as described herein can be conjugated to a detectable label and the binding of a detection reagent to asparaginase can be determined based on the intensity of the signal released from the detectable label. Alternatively, a secondary antibody specific to the detection reagent can be used. One or more antibodies may be coupled to a detectable label. Any suitable label known in the art can be used in the assay methods described herein. In some embodiments, a detectable label comprises a fluorophore. As used herein, the term “fluorophore” (also referred to as “fluorescent label” or “fluorescent dye”) refers to moieties that absorb light energy at a defined excitation wavelength and emit light energy at a different wavelength. In some embodiments, a detection moiety is or comprises an enzyme. In some embodiments, the enzyme (e.g., β-galactosidase) produces a colored product from a colorless substrate.

It will be apparent to those of skill in the art that this disclosure is not limited to immunoassays. Detection assays that are not based on an antibody, such as mass spectrometry, are also useful for the detection and/or quantification of asparaginase as provided herein. Assays that rely on a chromogenic substrate can also be useful for the detection and/or quantification of asparaginase as provided herein.

Alternatively, a level of a nucleic acid (e.g., DNA or RNA) encoding asparaginase in a sample can be measured via any method known in the art. In some embodiments, measuring the level of a nucleic acid encoding asparaginase comprises measuring mRNA. In some embodiments, the expression level of mRNA encoding asparaginase can be measured using real-time reverse transcriptase (RT) Q-PCR or a nucleic acid microarray. Methods to detect nucleic acid sequences include, but are not limited to, polymerase chain reaction (PCR), reverse transcriptase-PCR (RT-PCR), in situ PCR, quantitative PCR (Q-PCR), real-time quantitative PCR (RT Q-PCR), in situ hybridization, Southern blot, Northern blot, sequence analysis, microarray analysis, detection of a reporter gene, or other DNA/RNA hybridization platforms.

In some embodiments, an assay method described herein is applied to measure a level of methylation, for example, methylation of nucleic acids encoding asparaginase in cells contained in a sample. Such cells may be collected via any method known in the art and the level of methylation can be measured via any method known in the art, for example, sodium bisulfite conversion and sequencing.

Any binding agent that specifically binds to asparaginase may be used in the methods and kits described herein to measure the level of asparaginase in a sample. In some embodiments, the binding agent is an antibody or an aptamer that specifically binds to asparaginase protein. In other embodiments, the binding agent may be one or more oligonucleotides complementary to nucleic acids encoding asparaginase or a portion thereof. In some embodiments, a sample may be contacted, simultaneously or sequentially, with more than one binding agent that binds asparaginase protein and/or nucleic acids encoding asparaginase.

To measure the level of asparaginase, a sample can be in contact with a binding agent under suitable conditions. In general, the term “contact” refers to an exposure of the binding agent with the sample or cells collected therefrom for a suitable period of time sufficient for the formation of complexes between the binding agent and asparaginase in the sample, if any. In some embodiments, the contacting is performed by capillary action in which a sample is moved across a surface of a support membrane.

In some embodiments, the assays may be performed on low-throughput platforms, including single assay format. For example, a low throughput platform may be used to measure the presence and/or amount of asparaginase protein in biological samples (e.g., biological tissues, tissue extracts) for diagnostic methods, monitoring of disease and/or treatment progression, and/or predicting whether a disease or disorder may benefit from a particular treatment.

In some embodiments, a binding agent may be immobilized to a support member. Methods for immobilizing a binding agent will depend on factors such as the nature of the binding agent and the material of the support member and may utilize particular buffers. Such methods will be evident to one of ordinary skill in the art.

The type of detection assay used for detection and/or quantification of asparaginase such as those provided herein will depend on the particular situation in which the assay is to be used (e.g., clinical or research applications), and on what is being detected (e.g., protein and/or nucleic acids), and on the kind and number of patient samples to be run in parallel. The assay methods described herein may be used for both clinical and non-clinical purposes.

A level of asparaginase in a sample as determined by assay methods described herein, or any other assays known in the art, may be normalized by comparison to a control sample or a predetermined reference level to obtain a normalized value. A deviated level (e.g., increased or decreased) of asparaginase in a sample obtained from a subject relative to the level of asparaginase in a control sample or a predetermined reference level can be indicative of the presence of stomach cancer or liver cancer in the sample. In some embodiments, such a sample indicates that the subject from which the sample was obtained may have or be at risk for stomach cancer or liver cancer.

In some embodiments, a level of asparaginase in a sample obtained from a subject can be compared to a level of asparaginase in a control sample or predetermined reference level, and a deviated (e.g., increased or decreased) level of asparaginase may indicate that the subject has or is at risk for stomach cancer or liver cancer.

In some embodiments, a level of asparaginase in a sample obtained from a subject can be compared to a level of asparaginase in a control sample or predetermined reference level, and a deviated (e.g., increased or decreased) level of asparaginase may indicate that the subject is a candidate for asparaginase treatment as described herein.

A control sample may be a biological sample obtained from a healthy individual. Alternatively, a control sample may be a sample that contains a known amount of asparaginase. In some embodiments, a control sample is a biological sample obtained from a control subject. A control subject may be a healthy individual, e.g., an individual that is apparently free of stomach cancer or liver cancer, has no history of stomach cancer or liver cancer, and/or is undiagnosed with stomach cancer or liver cancer. A control subject may also represent a population of healthy subjects, e.g., a population of individuals that are apparently free of stomach cancer or liver cancer, have no history of stomach cancer or liver cancer, and/or are undiagnosed with stomach cancer or liver cancer.

A control sample may be used to determine a predetermined reference level. A predetermined reference level can represent a level of asparaginase in a healthy individual, e.g., an individual that is apparently free of stomach cancer or liver cancer, has no history of stomach cancer or liver cancer, and/or is undiagnosed with stomach cancer or liver cancer. A predetermined reference level can also represent a level of asparaginase in a population of subjects that do not have or are not at risk for stomach cancer or liver cancer (e.g., the average level in a population of healthy subjects). In other embodiments, a predetermined reference level can represent a level of asparaginase in a population of subjects that have stomach cancer or liver cancer.

A predetermined reference level can represent an absolute value or a range, determined by any means known to one of ordinary skill in the art. A predetermined reference level can take a variety of forms. For example, it can be single cut-off value, such as a median or mean. In some embodiments, such a predetermined reference level can be established based upon comparative groups, such as where one defined group is known to have stomach cancer or liver cancer and another defined group is known to not have stomach cancer or liver cancer. Alternatively, a predetermined reference level can be a range, for example, a range representing a level of asparaginase in a control population.

A predetermined reference level as described herein can be determined by methods known in the art. In some embodiments, a predetermined reference level can be obtained by measuring asparaginase levels in a control sample. In other embodiments, levels of asparaginase can be measured from members of a control population and the results can be analyzed by, e.g., by a computational program, to obtain a predetermined reference level that may, e.g., represent the level of asparaginase in a control population.

### General Techniques

The practice of the present disclosure will employ, unless otherwise indicated, conventional techniques of molecular biology (including recombinant techniques), microbiology, cell biology, biochemistry and immunology, which are within the ordinary skill in the art (Molecular Cloning: A Laboratory Manual, fourth edition (Green, et al., 2012 Cold Spring Harbor Press); Oligonucleotide Synthesis (M. J. Gait, ed., 1984); Methods in Molecular Biology, Humana Press; Cell Biology: A Laboratory Notebook, Vol. 3 (J. E. Cellis, ed., 2005) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Short Protocols in Molecular Biology (F. M. Ausubel, et al., eds., 2002); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). It is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the systems and methods provided herein and are not to be construed in any way as limiting their scope.

### Example 1: Profiling Metabolites from Cultured CCLE Cell Lines

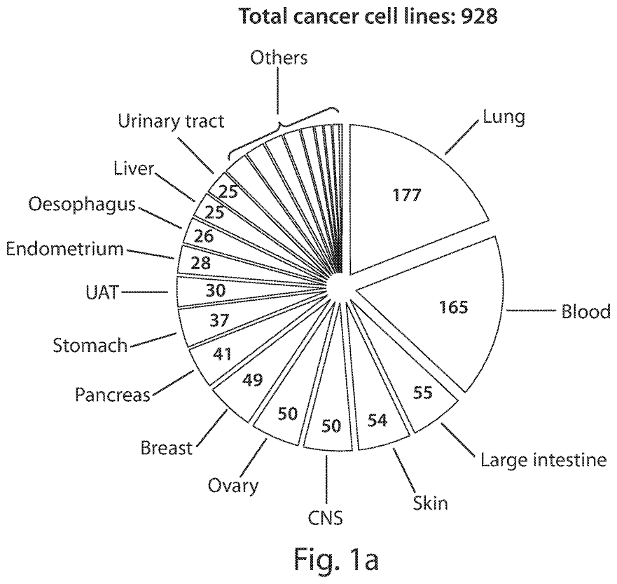

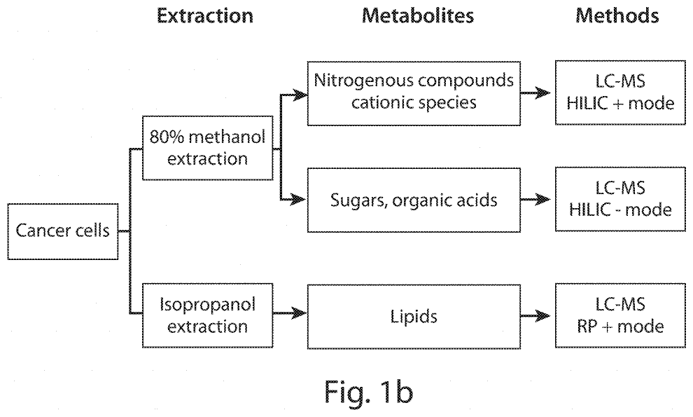

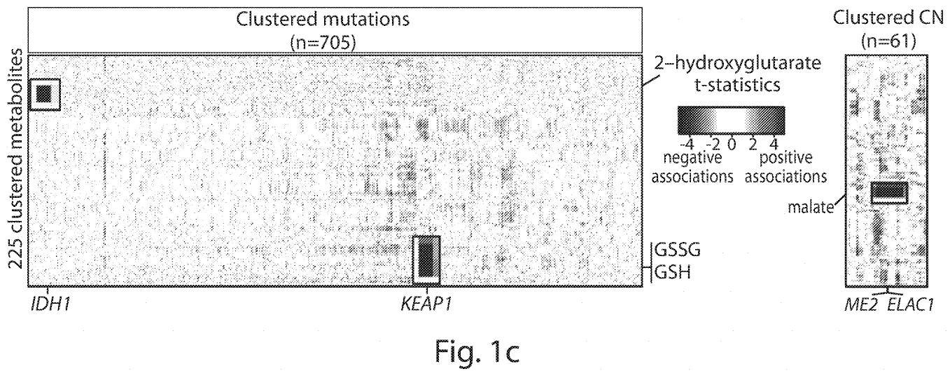

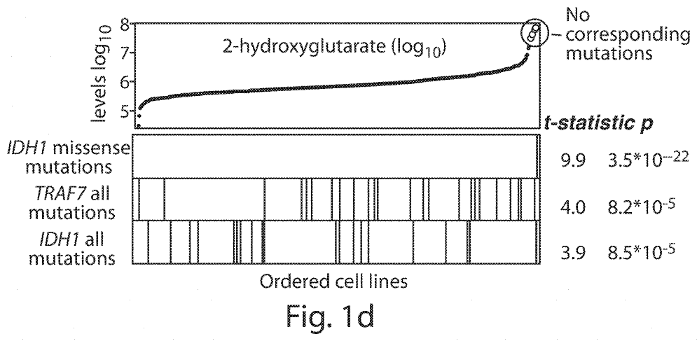

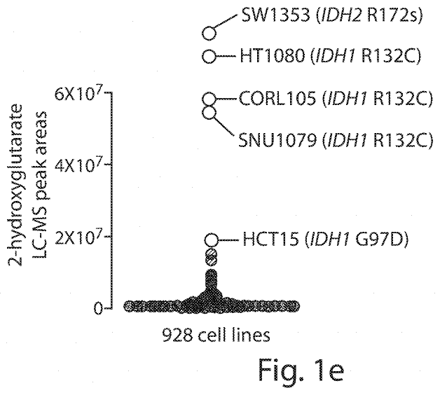

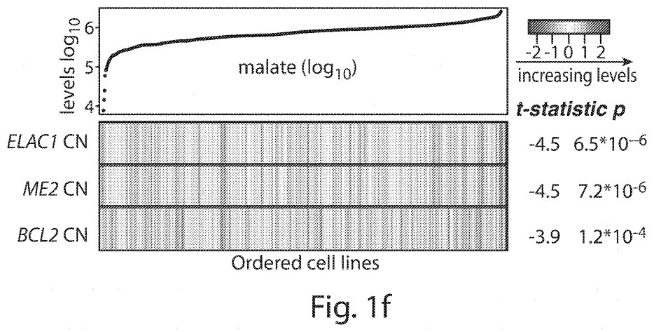

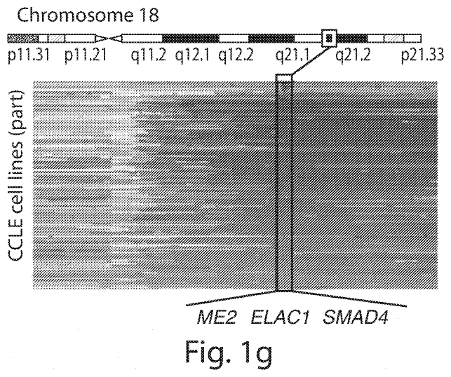

928 cancer cell lines from 20 major cancer types were cultured in vitro for metabolomic profiling of 124 polar and 101 lipid species (FIG. 1 (a)). Extracted polar and lipid metabolites were analyzed using hydrophilic interaction chromatography (HILIC) and reversed phase (RP) chromatography (FIG. 1 (b)). Sample measurements were obtained in four batches using pooled lysates as references to ensure consistent data quality. Trend normalization methods were applied before performing global comparisons.

### Example 2: Interrogating Metabolite Associations with Genetic Features

In addition to lineage, genetic or epigenetic events in cancer are likely to alter cellular metabolism. In order to identify metabolic variation that might be attributable to genetic differences, a matrix of genetic features was curated, including 705 gene mutations and 61 amplifications or deletions. To look for associations between these genetic features and metabolite levels, linear regression models controlling for lineage effects were applied (FIG. 1 (c)). The genetic features were scored by associations with each metabolite and can be compared in the order of statistical significance. Interestingly, it was found that mechanistically relevant features often displayed strong correlations with aberrant metabolite levels. Examples are discussed below.

First, unbiased comparison revealed the expected finding that for 2-hydroxyglutarate (2HG), the IDH1 hotspot missense mutation was a top predictive genetic feature (FIG. 1 (d)). Cell lines with an aberrant accumulation of this metabolite are mostly IDH1/IDH2 mutants (FIG. 1 (e)), recapitulating the known relationship^{9,10}. Notably, although there are no known IDH1/IDH2 mutants in the CCLE renal cell carcinoma lines (RCC), additional lineage effect analysis revealed that on average RCC cells had a 3-fold higher level of 2HG than others. This is consistent with the observation of increased 2HG levels in RCC tumors¹¹.

In copy-number space, using malate as an example, it was shown that the most strongly associated features are deletions of ELAC1 and ME2 (FIG. 1 (f)). These genes are co-localized in a 0.4 Mb region surrounding the tumor suppressor gene SMAD4 on chromosome 18 and are frequently co-deleted (FIG. 1 (g)). ME2 (malic enzyme 2) catalyzes the oxidative decarboxylation of malate to pyruvate.

To summarize, the resource described herein enables unbiased association analysis between metabolites and various genetic features and confirms previous findings linking oncogenic changes (e.g., IDH1/KEAP1/ME2) to aberrant metabolite levels.

### Example 3: DNA Methylation Regulates Metabolite Abundances

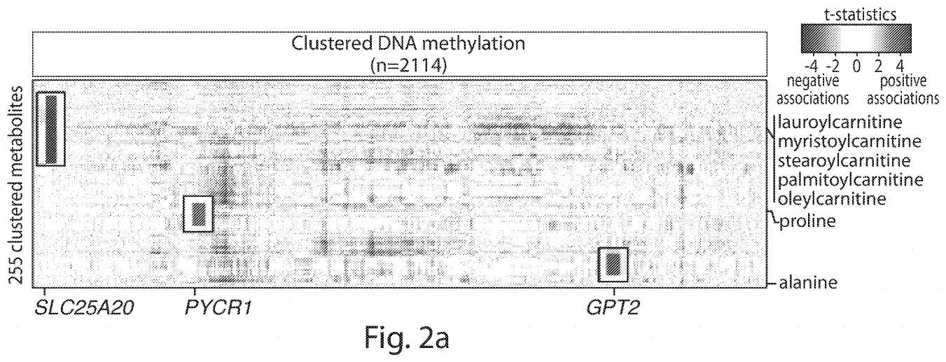

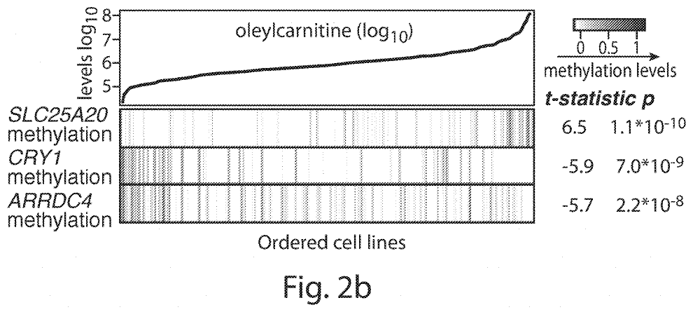

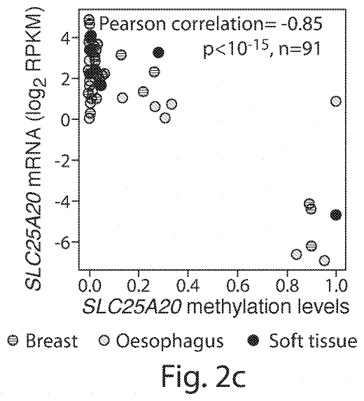

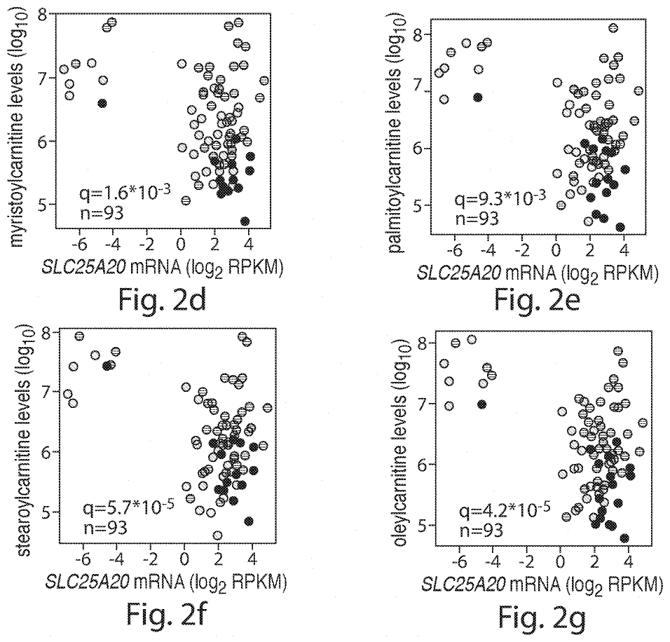

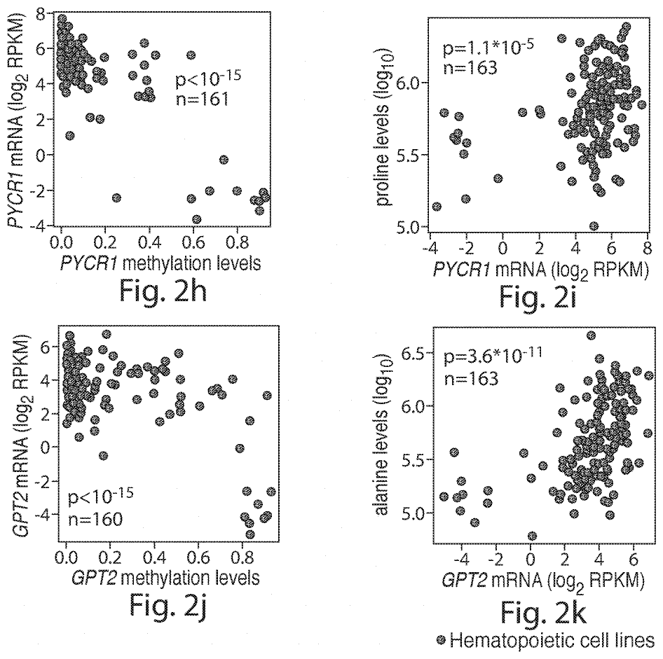

Next, DNA methylation was examined and the associations with the metabolite levels were assessed. 2114 genes whose mRNA transcripts were significantly associated with their promoter CpG methylation levels were included in this analysis given that these selected genes were likely to be regulated via DNA methylation. Systematic analysis of the correlates revealed a surprising number of specific alterations related to potential metabolic dysregulation (FIG. 2 (a)). These observations can be classified into two classes. First, DNA hypermethylation appears to influence metabolite levels via suppressing certain metabolite degradation pathways. For example, SLC25A20 methylation was strongly correlated with the accumulation of long-chain acylcarnitine species (e.g., oleylcarnitine) (FIG. 2 (b)). SLC25A20, also known as carnitine/acylcarnitine translocase, shuttles acylcarnitines across the mitochondrial inner membrane for fatty acid oxidation¹⁶. SLC25A20 hypermethylation correlated with marked mRNA transcript reduction (FIG. 2 (c)), which was associated with significantly elevated levels of acylcarnitine species having acyl chains of 14, 16 or 18 carbons (FIG. 2 (d-g)), indicating an unusual specific fatty acid catabolism defects in these cell lines. Second, DNA hypermethylation appears to regulate metabolite levels by limiting components of biosynthetic pathways. For example, reduced proline levels were associated with the hypermethylation of PYCR1, an enzyme that converts pyrroline-5-carboxylate to proline (FIG. 2 (h, i)). Additionally, decreased alanine levels were associated with the hypermethylation of GPT2, which can synthesize alanine via transamination (FIG. 2 (j, k)). Both of these effects were particularly strong among hematopoietic cell lines. Taken together, this resource provides an unbiased way to assess the impact of DNA methylation events in regulating intracellular metabolite concentrations.

### Example 4: Metabolite-Dependency Association Analysis

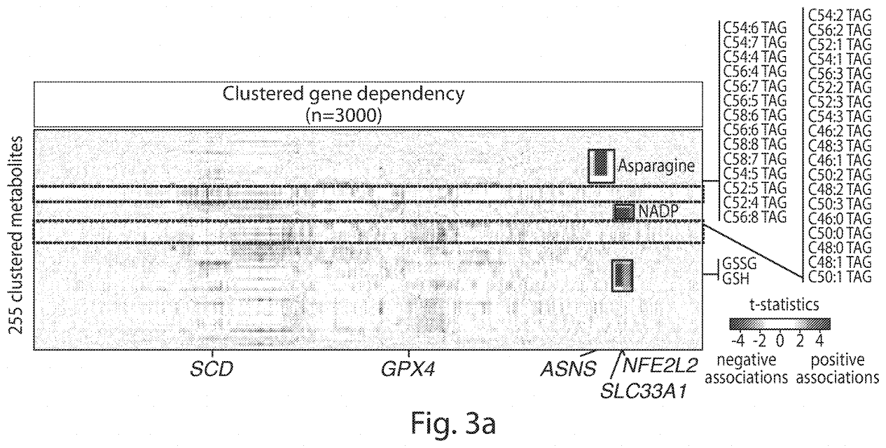

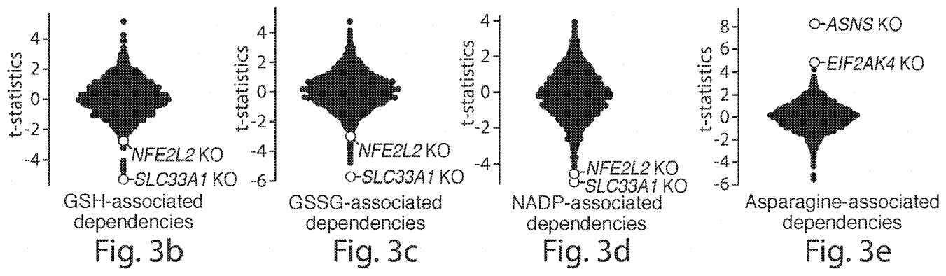

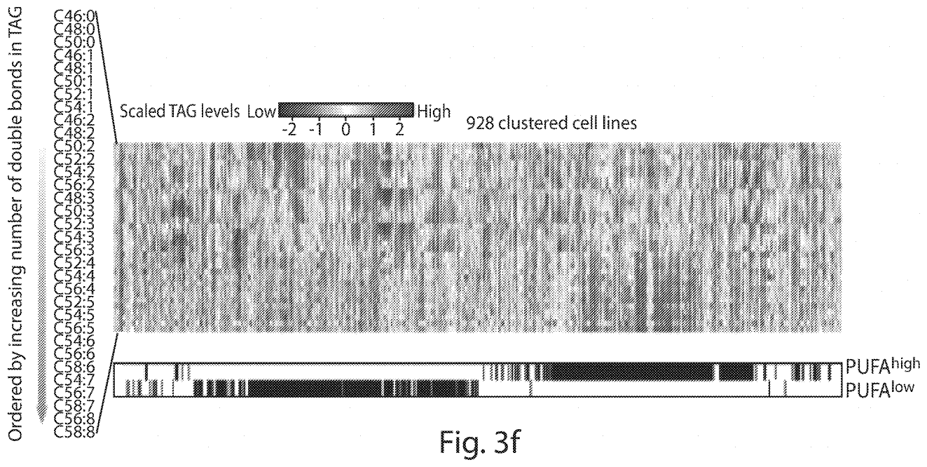

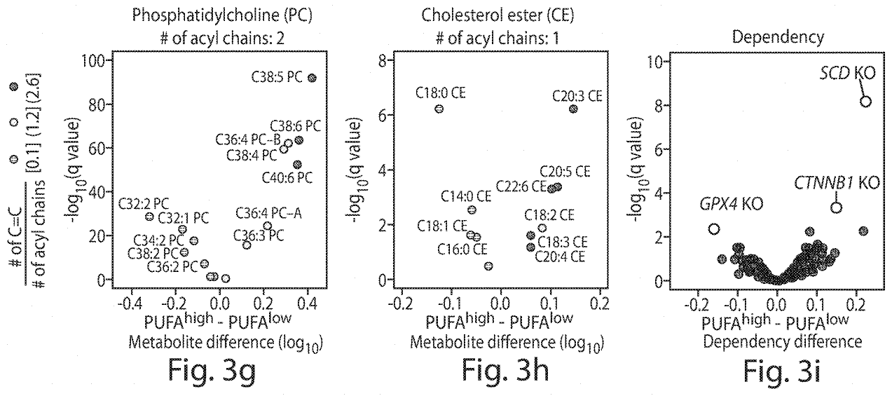

There has been a longstanding desire to take therapeutic advantage of dysregulated cancer metabolic states. To this end, a potential link was investigated between metabolic alterations to cancer vulnerabilities unveiled in the DepMap CRISPR-Cas9 knockout dataset in which 483 CCLE cell lines have been screened with a library of ˜74 k sgRNAs targeting ˜17,000 genes¹⁵. CERES scores were used to summarize gene-level dependency (small values indicate greater sensitivity to gene knockout)¹⁵ and then each gene level dependence was queried with respect to metabolite alterations. This unbiased metabolite-dependency association analysis shows that the dissimilar metabolic phenotypes observed in cancer cell lines are paired with distinct gene dependencies and therefore potential therapeutic targets (FIG. 3 (a)). Here, the study focused on the top 3000 dependent genes and highlights representative examples in metabolism related to redox balance, amino acids, and lipids. First, aberrant accumulation of redox metabolites including GSH, GSSG, and NADP⁺ (partly attributed to KEAP1 mutation, vide supra) was associated with increased sensitivity to knockout of NFE2L2 (NRF2), a transcription activator involved in antioxidant response (FIG. 3 (b-d)). Notably, the most associated dependency was SLC33A1 (FIG. 3 (b-d)), an acetyl-CoA transporter whose role in redox homeostasis is currently unknown. As another example, it was found that cells with lower asparagine levels were more dependent on its synthetase (ASNS) and EIF2AK4 (GCN2, involved in amino acid starvation response) (FIG. 3 (e)). Furthermore, an interesting association was also observed involving two distinct triacylglycerol (TAG) clusters (FIG. 3 (a)). One cluster consisted of polyunsaturated TAG species (at least 4 total C═C double bonds from 3 acyl chains) and the other cluster consisted of less unsaturated TAG species including monounsaturated fatty acyls (MUFA) (FIG. 3 (a)). To classify cancer cell lines enriched with either cluster, they were labeled as polyunsaturated fatty acyl high (PUFA^{high}, n=315) or polyunsaturated fatty acyl low (PUFA^{low}, n=325) after excluding those with non-significant lipid unsaturation differences (FIG. 3 (f)). This unsaturation difference also existed in other lipid species such as phosphatidylcholines (PC, FIG. 3 (g)), and cholesterol esters (CE, FIG. 3 (h)). To determine whether this distinct lipid utilization pattern might link to targetable dependencies, CERES scores were compared. It was found that the PUFA^{high} cell lines are sensitive to the knockout of GPX4 (FIG. 3 (i)), which mediates the detoxification of peroxidized PUFA¹⁷. In contrast, PUFA^{low} cell lines are sensitive to the loss of CTNNB1 or SCD (FIG. 3 (i)), which synthesizes MUFA. Together, these unbiased association analyses suggest that cancer cell lines cultured in vitro have significant lipidomic differences that can be selectively targeted based on PUFA classifications.

### Example 5: Phenotypic Profiling of Barcoded CCLE Lines

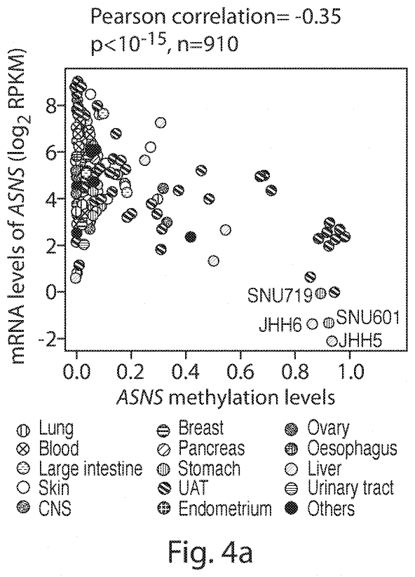

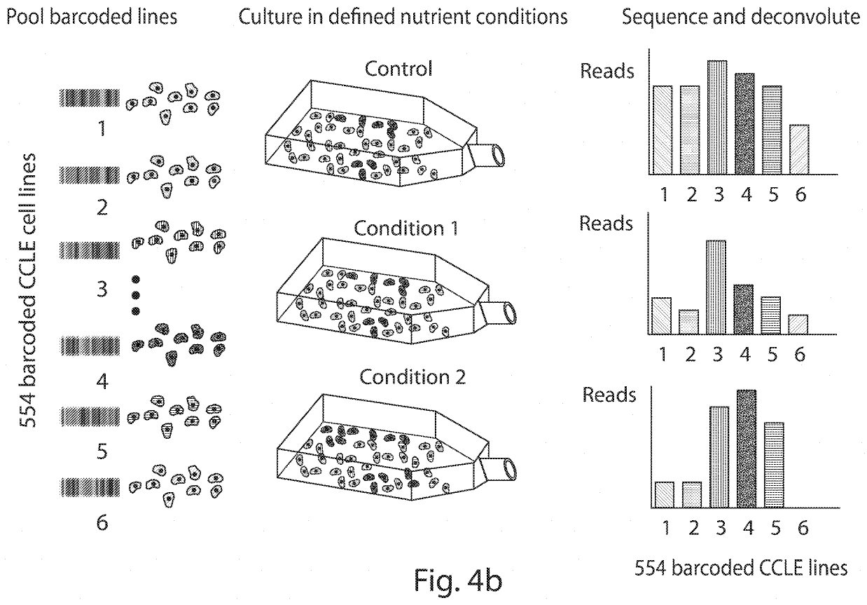

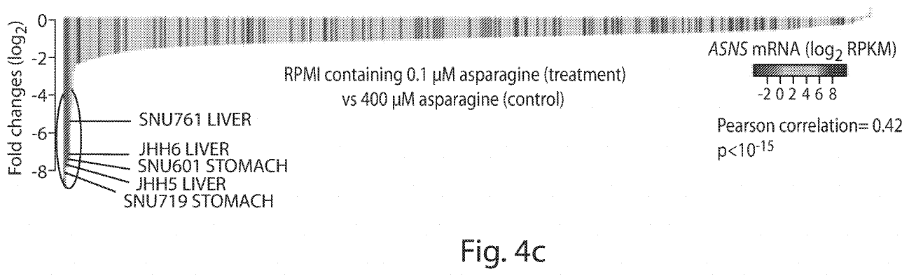

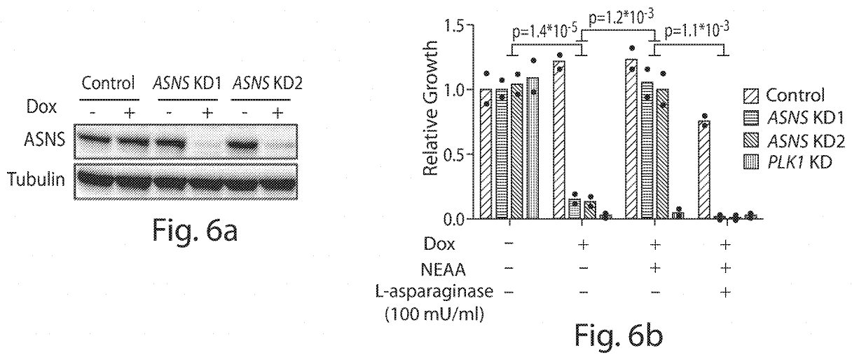

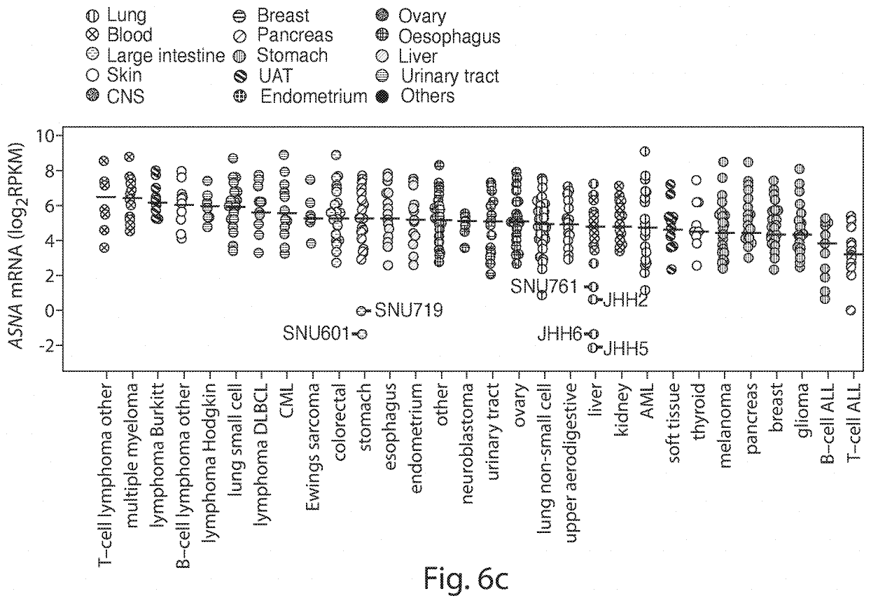

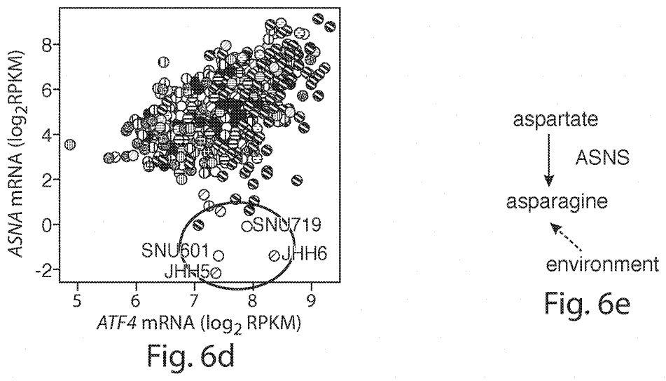

As shown in the results described herein, lower asparagine levels strongly associated with increased sensitivity to loss of asparagine synthetase (ASNS) (FIG. 3 (e)). The non-essential amino acid asparagine is synthesized by ASNS but can also be imported directly from the media. Studies herein showed that ASNS knockdown significantly impeded cell proliferation when media asparagine was limiting (FIG. 6 (a, b)). Given that some CCLE cell lines with ASNS promoter hypermethylation have aberrantly low ASNS expression even in the presence of its transcriptional activator ATF4 (FIG. 4 (a), FIG. 6 (c, d)), it was tested whether intrinsic methylation-dependent gene suppression might be selectively targeted using specific nutrient deprivation. To explore this, a variation of the PRISM technology where 544 adherent CCLE lines labeled with 24-nucleotide barcodes were grown in a pooled format¹⁸ (FIG. 4 (b)). The mixed cell pools were cultured under specific media conditions with defined amino acid concentrations and relative cell viability was then estimated by high-throughput sequencing of the barcode collected after 6 days of treatment. Here, we found that when the pooled cell populations were grown under limiting asparagine conditions, those with aberrantly low expression of ASNS were selectively depleted (FIG. 4 (c)). These examples suggest that DNA hypermethylation influences dependency on nutrient availability as exemplified by asparagine auxotrophy in subsets of cancer cell lines.

### Example 6: Expanding the Therapeutic Use of Asparaginase

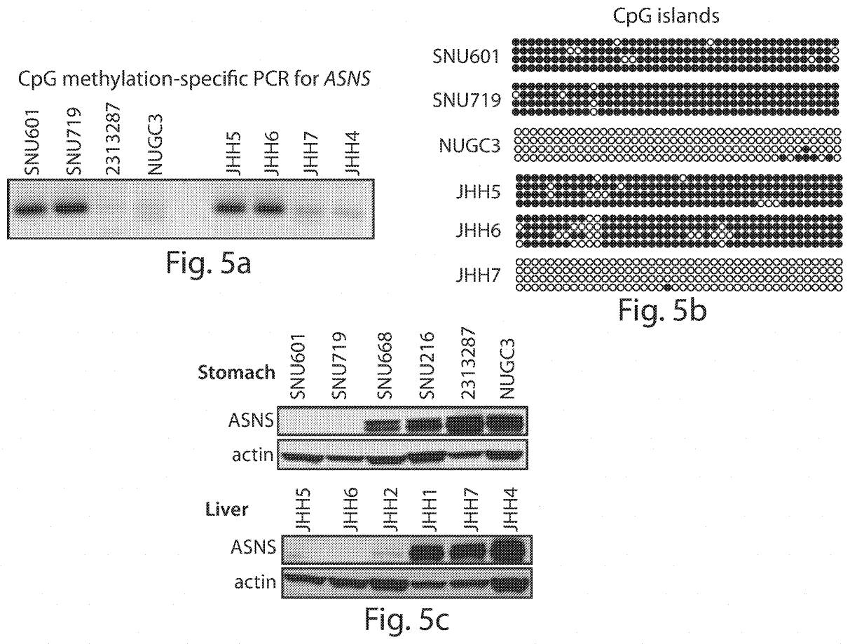

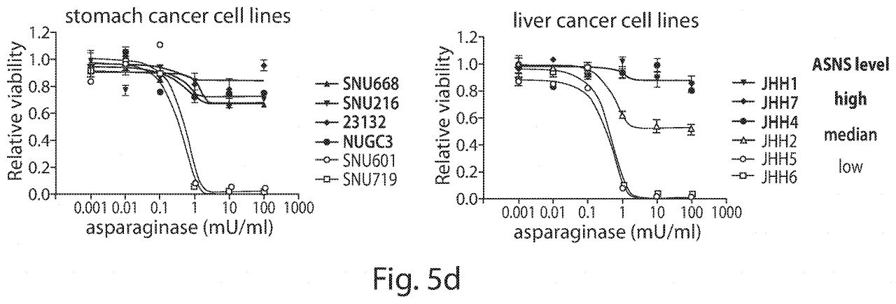

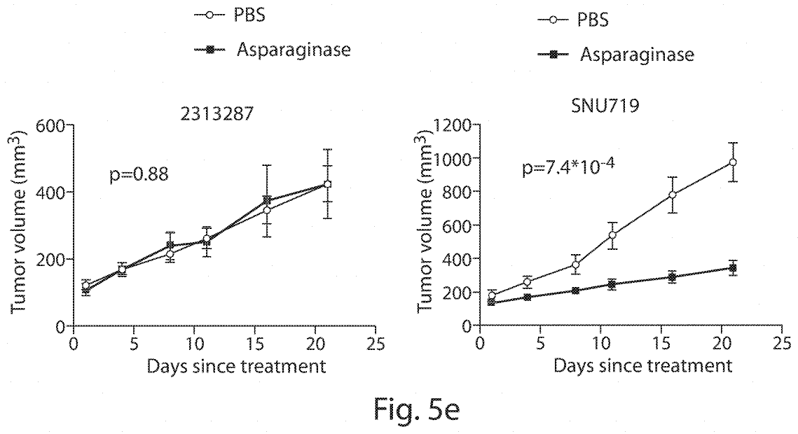

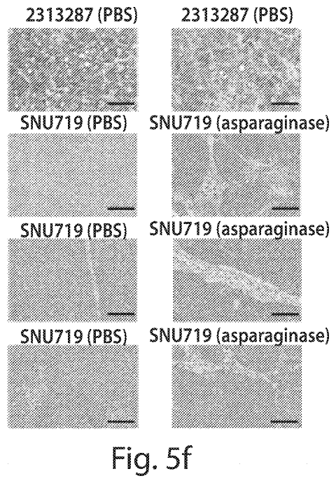

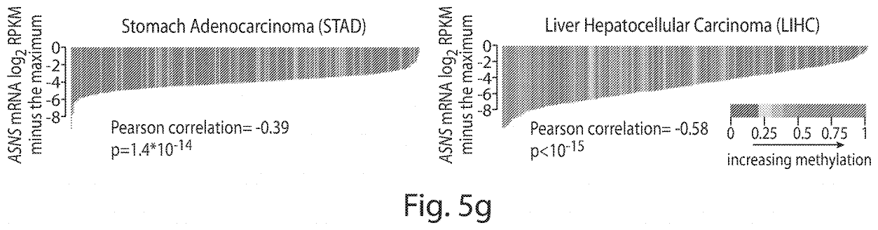

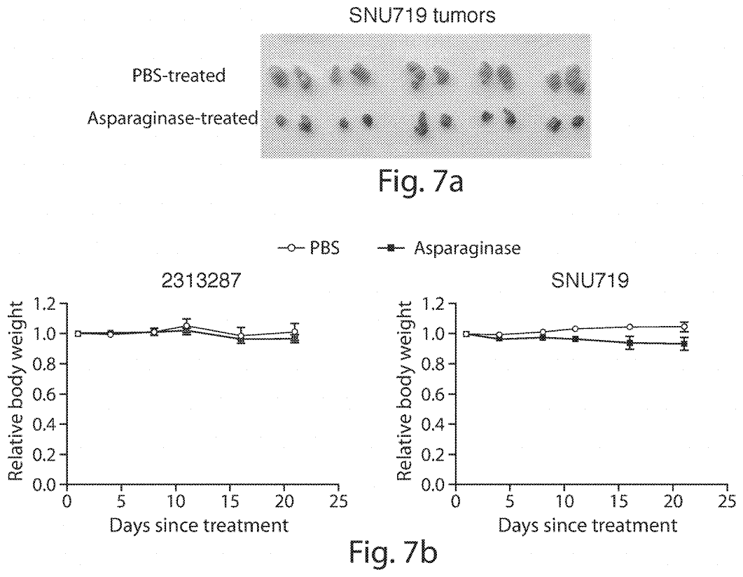

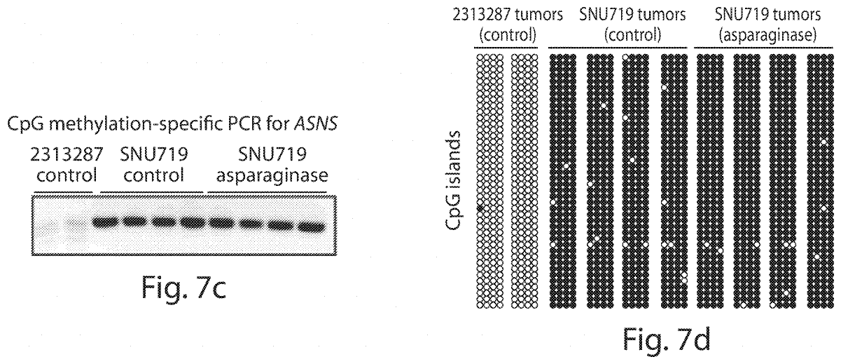

Nearly binary differences to asparagine depletion between cell lines with intrinsic lower expression of ASNS and the non-sensitive lines (FIG. 4 (c)) prompted exploration of the potential therapeutic value of asparaginase beyond its use in treating acute lymphoblastic leukemia (ALL). It was confirmed that cells with ASNS hypermethylation also lacked protein expression (FIG. 5 (a-c)) and were profoundly sensitive to asparaginase in vitro (FIG. 5 (d)). To determine whether this dependence could be reproduced in vivo, 7*10⁶ of U.S. Pat. No. 2,313,287 (ASNS high) or SNU719 (ASNS low) cells were subcutaneously implanted into both flanks of nude mice. After the tumors reached about 100-200 mm³ in volume, the mice were then treated with intraperitoneal injections of asparaginase (3000 units/kg/injection, 5 times a week) or vehicle control and monitored the tumor growth over a 3-week period. Here, a significant decrease of growth for SNU719 tumors but not 2313287 tumors with little body weight loss was observed (FIG. 5 (e), FIG. 7 (a, b)). It was also shown that ASNS hypermethylation and loss of expression was maintained during implantation and treatment of these xenografts (FIG. 5 (f), FIG. 7 (c, d)). These data also suggest that ASNS IHC might be applied to stratify and select patients for asparaginase trials. To define the relevant patient population based on data from human tumor samples, DNA methylation among gastric and hepatic cancers in The Cancer Genome Atlas (TCGA) was examined. Results showed significant association with reduced ASNS expression in tumor samples (FIG. 5 (g)). Collectively, these results suggest that asparaginase can suppress the growth of defined subsets of cancer cell lines with loss of ASNS expression both in vitro and in vivo.

### Example 8: Materials and Methods

Cell lines and culture conditions. Human cancer cell lines were collected as described previously. SNP genotyping was incorporated at each stage of cell culture to validate the identity of cell lines. The associated tissue type and gender information was annotated based on literature or vendor information when available. All cell lines were grown in T75 flasks with respective media using standard cell culture conditions (37° C., 5% CO₂) and were free of microbial contamination including mycoplasma. For each actively growing cell line with a low passage number, two million cells were seeded per T75 flask, the metabolites were extracted after 2 days and before the cells reached a confluence of 90%. Separate flasks were used for polar metabolite or lipid extractions.

Polar metabolite extraction. LC-MS grade solvents were used for all of the metabolite extraction in this study. For adherent cells, the media were aspirated off as much as possible and the cells were washed with 4 mL cold Phosphate Buffered Saline (PBS, no Mg²⁺/Ca²⁺). After vacuum aspiration of PBS, the metabolites were extracted by adding 4 mL 80% methanol (−80° C.) immediately and the samples were transferred to a −80° C. freezer. The flasks were kept on dry ice during the transfer and were incubated at −80° C. for 15 min. Then the lysate was collected by a cell scraper and transferred to a 15 mL conical tube on dry ice. The insoluble debris was removed by centrifuging at 3500 rpm for 10 min (4° C.). The supernatant was transferred to a new 15 mL conical tube on dry ice and the tube with the pellet was kept for further extraction. Then, 500 μL 80% methanol (−80° C.) was added to each pellet. The mixture was resuspended by vortexing or pipetting and transferred to a 1.5 ml centrifuge tube on dry ice. The cell debris was removed by centrifuging samples at 10,000 rpm for 5 min (4° C.). The supernatant was transferred to the corresponding 15 mL conical tube on dry ice so that all extracts were combined. The pooled extracts were stored at −80° C. before LC-MS analysis.

For cells growing in suspension, they were centrifuged to pellet at 300 g for 5 min (4° C.) and the supernatant was then aspirated off as much as possible. These cells were washed once with 4 mL cold PBS (no Mg²⁺/Ca²⁺) and they were pelleted at 300 g for 5 min (4° C.). After vacuum aspiration of PBS, the metabolites were extracted by adding 4 mL 80% methanol (−80° C.) immediately and the samples were transferred to a −80° C. freezer after brief vortexing. The samples were kept on dry ice during the transfer and were incubated at −80° C. for 15 min. The insoluble debris was removed by centrifuging at 3500 rpm for 10 min (4° C.). The subsequent steps were the same as those used for adherent cell lines.

Lipid extraction. For adherent cells, the medium was aspirated off as much as possible and the cells were washed with 4 mL cold PBS (no Mg²⁺/Ca²⁺). After vacuum aspiration of PBS, the lipid metabolites were extracted by adding 4 mL isopropanol (4° C.) and the lysate was collected by a cell scraper and transferred to a 15 mL conical tube on ice. The samples were covered to avoid exposure to light and were allowed to sit for 1 h at 4° C. Samples were then vortexed and the cell debris was removed by centrifuging at 3500 rpm for 10 min (4° C.). The supernatant was transferred to a new 15 mL centrifuge tube on ice and stored at −20° C. before LC-MS analysis.

For cells growing in suspension, they were centrifuged to pellet at 300 g for 5 min (4° C.) and the supernatant was then aspirated off as much as possible. These cells were washed once with 4 mL cold PBS (no Mg²⁺/Ca²⁺) and they were pelleted at 300 g for 5 min (4° C.). After vacuum aspiration of PBS, the lipid metabolites were extracted by adding 4 mL isopropanol (4° C.) immediately. After brief vortexing, the samples were covered to avoid exposure to light and were allowed to sit for 1 h at 4° C. The insoluble debris was removed by centrifuging at 3500 rpm for 10 min (4° C.). The supernatant was transferred to a new 15 mL centrifuge tube on ice and stored at −20° C. before LC-MS analysis.

LC-MS instrumentation and methods. A combination of two hydrophilic interaction liquid chromatography (HILIC) methods, either acidic HILIC method with positive-ionization-mode MS, or basic HILIC method with negative-ionization-mode MS was used to profile polar metabolites. Reversed Phase (RP) chromatography was used to profile lipid species. The LC-MS methods were based on a previous study²⁸, where the metabolite retention time and the selected reaction monitoring parameters were also described. LC-MS related reagents were purchased from Sigma-Aldrich if not specified. Pooled samples composed of 11 cell lines from different lineages were used for trend and batch correction.

The LC-MS system for the first method consisted of a 4000 QTRAP triple quadrupole mass spectrometer (SCIEX) coupled to an 1100 series pump (Agilent) and an HTS PAL autosampler (Leap Technologies). Polar metabolite extracts were reconstituted with acetonitrile/methanol/formic acid (74.9:24.9:0.2 v/v/v) containing stable isotope-labeled internal standards (0.2 ng/μL valine-d8 (Isotec) and 0.2 ng/μL phenylalanine-d8 (Cambridge Isotope Laboratories)). The samples were centrifuged (10 min, 9,000 g, 4° C.), and the supernatants (10 μL) were injected onto an Atlantis HILIC column (150×2.1 mm, 3 μm particle size; Waters Inc.). The column was eluted isocratically at a flow rate of 250 μL/min with 5% mobile phase A (10 mM ammonium formate and 0.1% formic acid in water) for 1 min followed by a linear gradient to 40% mobile phase B (acetonitrile with 0.1% formic acid) over 10 min. The ion spray voltage was set to be 4.5 kV and the source temperature was set to be 450° C.

The second method using basic HILIC separation and negative ionization mode MS detection was established on an LC-MS system consisting of an ACQUITY UPLC (Waters Inc.) coupled to a 5500 QTRAP triple quadrupole mass spectrometer (SCIEX). Polar metabolite extracts spiked with the isotope labeled internal standards including 0.05 ng/μL inosine-¹⁵N₄, 0.05 ng/μL thymine-d4, and 0.1 ng/μL glycocholate-d4 (Cambridge Isotope Laboratories) were centrifuged (10 min, 9,000 g, 4° C.), and 10 μL supernatants were injected directly onto a Luna NH2 column (150×2.0 mm, 5 μm particle size; Phenomenex) that was eluted at a flow rate of 400 μL/min with initial conditions of 10% mobile phase A (20 mM ammonium acetate and 20 mM ammonium hydroxide in water (VWR) and 90% mobile phase B (10 mM ammonium hydroxide in 75:25 v/v acetonitrile/methanol (VWR)) followed by a 10-min linear gradient to 100% mobile phase A. The ion spray voltage was set to be −4.5 kV and the source temperature was set to be 500° C.

Lipids were profiled using a 4000 QTRAP triple quadrupole mass spectrometer (SCIEX) coupled to a 1200 Series Pump (Agilent Technologies) and an HTS PAL autosampler (Leap Technologies). Lipid extracts in isopropanol, spiked with an internal standard (0.25 ng/μL 1-dodecanoyl-2-tridecanoyl-sn-glycero-3-phosphocholine (Avanti Polar Lipids)), were centrifuged and 10 μL supernatants were injected directly to a 150×3.0 mm Prosphere HP C4 column (Grace) for reversed phase chromatography. Mobile phase A was 95:5:0.1 (v/v/v) 10 mM ammonium acetate/methanol/acetic acid. Mobile phase B was 99.9:0.1 (v/v) methanol/acetic acid. The column was eluted isocratically with 80% mobile phase A for 2 minutes, followed by a linear gradient to 80% mobile phase B over 1 minute, a linear gradient to 100% mobile phase B over 12 minutes, and then 10 minutes at 100% mobile phase B. MS analyses were carried out using electrospray ionization and performed in the positive-ion mode with Q1 scans. Ion spray voltage was set to be 5.0 kV, and the source temperature was set to be 400° C.

Generation of isogenic cell lines. A2058 cells were maintained in DMEM, supplemented with 10% FBS and 2 mM glutamine. 1% non-essential amino acids (NEAA, BioConcept, 5-13K00) was added if stated. This NEAA mix (100×) contained 10 mM of L-asparagine, L-alanine, L-aspartic acid, L-glutamic acid, L-proline, L-serine, and glycine. shRNA (Control_KD: AGAAGAAGAAATCCGTGTGAA (SEQ ID NO: 1), ASNS_KD1: GCATCCGTGGAAATGGTTAAA (SEQ ID NO: 2); ASNS_KD2: CATTCAGGCTCTGGATGAAGT (SEQ ID NO: 3); PLK1_KD: GGTATCAGCTCTGTGATAACA (SEQ ID NO: 4) were cloned in inducible pLKO-based lentiviral vectors (puromycin resistant). Wild type A2058 was infected with shRNA-expressing viruses respectively. After selection, the KD efficiency was evaluated by western blots upon 3 days of treatment with doxycycline (100 ng/mL).

Pooled screens of barcoded CCLE lines. The CCLE lines were barcoded and screened as described previously¹⁸. Briefly, cells were mixed as individual pools (˜24 lines in each) and kept frozen in liquid nitrogen before use. On the day of experiment, the individual pools were mixed together in corresponding media conditions with equal numbers so that each line started from about 200 cells per T25 flask. After 6 days, the genomic DNA was extracted and the barcodes were amplified by PCR before high-throughput sequencing. Three biological replicates were used in each condition and the growth changes were calculated with the control conditions as reference.

Animal studies. The animal work was approved by the Institutional Animal Care and Use Committee (IACUC) at the Broad Institute. 4-week-old, female, athymic nude mice (CrTac:NCr-Foxn1^{nu}, Taconic) were inoculated subcutaneously with 7*10⁶ cancer cells in phenol red free RPMI media with 50% matrigel in both flanks. The mice were randomized into treatment or control group when tumors reached approximately 100-200 mm³ in size. Asparaginase (Abcam) was delivered with intraperitoneal injection at 3000 units/kg in 200 μl PBS 5 times per week (omitting Wednesday and Sunday) for 3 weeks. Tumor tissues were collected and processed for IHC staining by standard methods. All IHC staining was performed on the Leica Bond automated staining platform. Polyclonal Asparagine Synthetase (ASNS) antibody from Proteintech (#14861-1-AP) was run at 1:1500 dilution using the Leica Biosystems Refine Detection Kit with citrate antigen retrieval. Tumor sizes were calculated by ½*length*width*width.

Analysis of DNA methylation. The CCLE reduced representation bisulfite sequencing (RRBS) data was used for gene methylation analysis. For independent validation and cell lines not covered (e.g., JHH5, JHH6), genomic DNA from cell line or tumor samples was isolated and bisulfite-converted using the EpiTect Fast LyseAll Bisulfite Kit (Qiagen) following manufacturer's instructions. For methylation-specific PCR, the primer set consisted of 5′CGTATTGAGACGTAAGGCGT3′ (SEQ ID NO: 5) and 5′CTAACTCCTATAACGCGTACGAAA3′ (SEQ ID NO: 6). For bisulfite sequencing, the primer set consisted of 5′GTTAGAATAGTAGGTAGTTTGGG3′ (SEQ ID NO: 7) and 5′AAAATACACATATAACATTTACAAAAACTC3′ (SEQ ID NO: 8). Purified PCR products were cloned into the pCR™4-TOPO® TA vector using TOPO TA Cloning Kit (Invitrogen).

Statistical analysis. All statistical analyses used in this paper were done in R v 3.4.2 (downloaded from www.r-project.org/). Data visualization was done in R and Prism (GraphPad). Statistics and relevant information including the type and the number of replicates (n), the adopted statistical tests, and p-values are reported in the figures and associated legends. For Pearson correlations, the cor.test function in R was used to conduct significance test and obtain the p-values (two-sided). The Benjamini-Hochberg procedure was used to control for multiple hypothesis testing when applicable.

Metabolite data acquisition and quality control. Raw data were processed using MultiQuant 1.2 software (SCIEX) for automated LC-MS peak integration. All chromatographic peaks were also manually reviewed for the quality of integration and compared against known standards for each metabolite to confirm identities. Internal standard peak areas were monitored for quality control and to assess system performance over time. Additionally, pooled samples composed of mixed metabolites from 11 cell lines (NCIH446, DMS79, NCIH460, DMS53, NCIH69, HCC1954, CAMA1, KYSE180, NMCG1, UACC257, and AU565) were used after every set of 20 samples. This was an extra quality control measure of analytical performance and also served as a reference for scaling raw metabolomic data across samples. The peak area for each metabolite in each sample was standardized by computing the ratio between the value observed in the sample and the value observed in the “nearest neighbor” pooled sample. These ratios were then multiplied by the mean value of all reference samples for each analyte to obtain standardized peak areas.

To remove potential batch effects, the ratio between the mean standardized peak area for each metabolite in a given batch and the mean standardized peak area for that metabolite across all the batches was computed. Then the standardized peak areas for that metabolite in that given batch were divided by that ratio. Note that the abundance of different metabolites cannot be compared given the nature of the LC-MS methods. Only for the same metabolite, the levels could be compared between different cell lines. The final batch-corrected standardized peak areas were then login-transformed. Additionally, considering the cell line to cell line variation in biomass that could contribute to systematic differences in metabolite abundance detected by LC-MS, the data was processed by two steps. First, each column of metabolites was calibrated to have the same median. Then each row (cell line) was calibrated to have the same median. Empirically, this median normalization step effectively calibrated metabolomic datasets, adjusting artificial differences due to different sample biomass before metabolite extraction.

Missing data handling. For the trend-corrected metabolomic dataset, a small fraction of values were missing. Imputations were first applied using fully conditional specification implemented by the Multivariate Imputation via Chained Equations (MICE) algorithm from R package “mice”, which has the advantage of preserving intrinsic data matrix structure and information. The quality of predictive-mean-matching-based imputations was inspected using diagnostic tools in the package. It was observed that the generated multiple matrices had negligible differences for most downstream applications due to the small fraction (9%) of missing values and the strong signals from observed values. Therefore, one representative imputed matrix was chosen for downstream regression analysis that required a complete data structure for efficient computation.

Other cancer cell line dataset acquisition. The CCLE datasets (e.g., mutation, copy number variation, RNAseq) were downloaded from the Broad Institute CCLE portal. The CRISPR-Cas9-based gene-essentiality data used (CERES scores, 2019Q1 release) were obtained from the Cancer Dependency Map project¹⁵.

Clustering and heatmap plotting. Clustering was done in R with the function hclust. Note that each feature (e.g., metabolite) was scaled to have mean 0 and standard deviation 1 before hierarchical clustering analysis and heatmap plotting. The dissimilarity was defined as 1 minus the Pearson correlation between each pair of selected features. The resulting distance matrix was processed by the “centroid” method in the hclust function to get the clustering results. For heatmap plots, the heatmap.2 function in the R package gplots was used.

Metabolite lineage effect analysis. To evaluate the association between the metabolite levels and the lineage types, a linear regression model was applied. The lineage types were coded as binary covariates (X). Cell lines were represented by the rows, with 1 indicating presence of the corresponding feature. Each metabolite level (log₁₀ scale) was used as the response variable Y. The calculated r² was used to characterize the lineage effects quantitatively.

Genetic, epigenetic, and dependency feature collection. Genetic and epigenetic features were curated in the association analysis with CCLE metabolites. These included all nonsynonymous mutations of 474 cancer-related genes, deleterious, loss-of-function mutations of 202 genes, and hotspot missense mutations of 29 genes (TCGA hotspot count >=10; portals.broadinstitute.org/ccle). Such discrete features were converted to binary indicators (1/0) in the analysis. 40 genes with frequent deletions and 21 genes with frequent amplifications were also selected. These copy number alteration events were validated to significantly associate with corresponding gene transcriptional levels (CCLE RNAseq data). Additionally, the methylation scores of 2,114 genes were included given their significant negative associations with the corresponding transcriptional levels (CCLE RNAseq data). To select dependencies, the focus was on the top 3,000 genes ordered by variance of CERES scores across the panel of cell lines. Genes with less cell-line-to-cell-line dependency difference (e.g., non-essential) were not prioritized for metabolite-dependency association analysis.

Linear regression analysis. A linear regression model was applied to evaluate associations between two different datasets of CCLE cell lines (e.g., genetic feature vs metabolite level). Lineage variables were included to account for lineage-associated confounding effects when cell lines from different lineages were analyzed together.

First, a covariate matrix was constructed with cell lines as rows and features as columns for the linear regression. In addition to the intercept variable I, binary variables indicating major lineages were also included. Here, L1, L2, . . . , L17 represented the lineages of lung, large intestine, blood, urinary, bone, skin, breast, liver, ovary, oesophagus, endometrium, central nervous system, soft tissue, pancreas, stomach, kidney, and upper aerodigestive tract. Further, variable (X) was added to this covariate matrix: each mutation variable was binary-coded; each continuous variable (e.g., mRNA log₂ RPKM) was rescaled to have mean 0 and standard deviation 1.

The dependent variable vector Y could be another type of cell features. The coefficient vector was represented as β. For example, to answer the question that in a given cell line feature matrix (e.g., collections of genetic or epigenetic features) which feature was the most associated with a given metabolite vector under the condition of controlled lineage effects, this regression analysis was applied to individual features (e.g., individual genetic and epigenetic features) before comparisons. The calculated t-statistics, p-values, and estimated coefficients for X (βx) were reported to evaluate the associations.

### Discussion

Despite considerable efforts to identify cancer metabolic alterations that might unveil druggable vulnerabilities, systematic characterizations of metabolism as it relates to functional genomic features and associated dependencies remain uncommon. To further understand the metabolic diversity in cancer, studies described herein profiled 225 metabolites in 928 cell lines from more than 20 cancer types in the CCLE using liquid chromatography-mass spectrometry (LC-MS). This resource enables unbiased association analysis linking cancer metabolome to genetic alterations, epigenetic features, and gene dependencies. Additionally, by screening barcoded cell lines, it was demonstrated that aberrant ASNS hypermethylation sensitizes subsets of gastric and hepatic cancers to asparaginase therapy. These findings and related methodology provide comprehensive resources that will help to clarify the landscape of cancer metabolism.

Cell metabolism involves a highly coordinated set of activities in which multi-enzyme systems cooperate to convert nutrients into building blocks for macromolecules, energy currencies, and biomass^{1,2}. In cancer, genetic or epigenetic changes can perturb the activity of key enzymes or rewire oncogenic pathways resulting in cell metabolism alterations^{3,4}. Specific metabolic dependencies in cancer have also been the basis for effective therapeutics including inhibitors that target IDH1, as well as folate and thymidine metabolism⁵. The search for new drug targets, however, has been hampered, at least in part, by the fact that cancer metabolomic studies often draw conclusions from small numbers of cell lines from which generalizations are difficult. In contrast, there have been no systematic profiling efforts that encompass hundreds of cellular and genetic contexts. Furthermore, there is no high-throughput methodology that assesses cancer metabolic needs by perturbing related pathways across many cell lines. Consequently, the discovery of new anticancer metabolic targets might benefit from high-quality, comprehensive metabolomic data in addition to the current CCLE-related characterization that includes genomic, transcriptomic features as well as genetic dependency maps⁶⁻⁸.

Cancers are diverse in histology, in the pattern of underlying genetic alterations, and in metabolic signatures. To date, there has been no systematic metabolomic profiling for hundreds of model cancer cell lines from multiple lineages with distinct genetic backgrounds. To bridge this gap, 225 metabolites in a collection of 928 cancer cell lines were profiled, and the resulting data was intersected with other large-scale profiling datasets. This breadth and depth allows for various metabolic signatures to be probed in an unbiased manner and for metabolites with similar patterns to be identified. Beyond the diversity revealed in baseline metabolite levels, the diverse proliferative responses to perturbations in the dynamic metabolic networks with pooled screens of 554 barcoded cell lines were also investigated. Overall, the data and analyses suggest that distinct metabolic phenotypes exist in cancer cell lines both at the unperturbed and the perturbed states and that such phenotypes have direct implications for therapeutics targeting metabolism.

In particular, prevalent DNA methylation events were delineated in addition to somatic mutations and copy number alterations in various metabolic pathways began to unveil their key regulatory roles both at the basal state and in the dynamics of cell growth. On one hand, gene hypermethylation events likely influence baseline metabolite abundance via reductions in key enzymes mediating metabolite degradation (e.g., SLC25A20 with long-chain acylcarnitines) or synthesis (PYCR1 with proline, GPT2 with alanine). Alternatively, methylation-dependent suppression of gene expression can have profound modulatory effects in cell proliferation under altered nutrient conditions (e.g., ASNS with asparagine).

Several observations described herein relate to potential therapeutic applications. The suppressed ASNS expression in subsets of stomach and liver cancers suggest the use of asparaginase as a therapeutic option for subpopulations in these diseases. Although asparaginase is an effective agent used in the regimen for ALL²⁵, there has been no evidence for its potential efficacy for solid tumors in the clinic. This is consistent with the observation of abundant ASNS baseline expression in most lineages except the ALL where expression of ASNS is low. This underlying intrinsic dependence sharply contrasts with the studies combining ASNS inhibition with asparagine depletion in solid tumors^{26,27}. Consequently, studies described herein relating to asparaginase use in treating solid tumors with intrinsic loss of ASNS may have therapeutic implications.

### Tables

TABLE 1
Cell culture media.

| Name | Vendor | Catalog number |
|---|---|---|
| DMEM/F-12 | Invitrogen | Cat# 11330-057 |
| DMEM | Invitrogen | Cat# 12430-062 |
| EMEM | ATCC | Cat# 30-2003 |
| Ham's F10 | Invitrogen | Cat# 11550-043 |
| Ham's F12 | Invitrogen | Cat# 11765-054 |
| IMDM | Invitrogen | Cat# 12440-053 |
| Leibovitz's L-15 | Invitrogen | Cat# 11415-064 |
| McCoy's 5A | Invitrogen | Cat# 16600-082 |
| MCDB 105 | Cell applications | Cat# 117-500 |
| Medium 199 | Invitrogen | Cat# 11150-059 |
| RPMI 1640 | Invitrogen | Cat# 22400-105 |
| Waymouth MB 7521 | Invitrogen | Cat# 11220-035 |
| Williams' E Medium | Invitrogen | Cat# 12551 |
| Fetal bovine serum (FBS) | ATCC | Cat# 30-2020 |
| Customized RPMI without | AthenaES | NA |
| specific components | | |

TABLE 2
Coefficient of variation (CV) for each metabolite.

| Metabolite | CV | Metabolite | CV | Metabolite | CV |
|---|---|---|---|---|---|
| C38:5 PC | 0.009 | methionine | 0.024 | Serine | 0.038 |
| C36:4 PC-B | 0.009 | phenylalanine | 0.024 | glucuronate | 0.038 |
| C38:6 PC | 0.009 | C16:0 SM | 0.024 | taurocholate | 0.038 |
| C34:1 PC | 0.010 | C32:2 PC | 0.024 | Urate | 0.038 |
| C38:4 PC | 0.010 | 3-methyladipate/pimelate | 0.024 | erythrose-4-phosphate | 0.038 |
| C36:4 PC-A | 0.011 | C56:6 TAG | 0.024 | C36:2 DAG | 0.039 |
| C36:2 PC | 0.013 | creatinine | 0.024 | Sarcosine | 0.039 |
| threonine | 0.013 | inositol | 0.024 | Citrate | 0.040 |
| glutamate | 0.013 | F1P/F6P/G1P/G6P | 0.024 | C46:1 TAG | 0.040 |
| C18:2 LPC | 0.013 | C50:3 TAG | 0.024 | hippurate | 0.040 |
| oxalate | 0.014 | pantothenate | 0.025 | C34:2 DAG | 0.040 |
| proline | 0.014 | succinate/methylmalonate | 0.025 | dCMP | 0.041 |
| C34:3 PC | 0.014 | hexoses (HILIC neg) | 0.025 | butyrobetaine | 0.041 |
| C36:1 PC | 0.015 | C58:8 TAG | 0.025 | 4-pyridoxate | 0.042 |
| isoleucine | 0.015 | phosphocreatine | 0.026 | cotinine | 0.043 |
| C22:6 LPC | 0.015 | C18:3CE | 0.026 | DHAP/glyceraldehyde 3P | 0.043 |
| glutamine | 0.015 | C20:3 CE | 0.026 | C56:7 TAG | 0.043 |
| C20:4 LPE | 0.015 | C46:2 TAG | 0.026 | hexoses (HILIC pos) | 0.044 |
| C32:1 PC | 0.015 | C16:1 LPC | 0.026 | GABA | 0.044 |
| C36:3 PC | 0.016 | methionine sulfoxide | 0.026 | NMMA | 0.045 |
| C38:2 PC | 0.016 | C32:0 PC | 0.026 | malondialdehyde | 0.045 |
| C34:2 PC | 0.016 | C24:0 SM | 0.026 | isocitrate | 0.046 |
| C54:6 TAG | 0.016 | SDMA/ADMA | 0.026 | oleylcarnitine | 0.046 |
| C22:1 SM | 0.016 | aspartate | 0.026 | alpha-hydroxybutyrate | 0.046 |
| xanthine | 0.017 | C46:0 TAG | 0.026 | xanthosine | 0.047 |
| C56:8 TAG | 0.017 | C52:5 TAG | 0.027 | 3-phosphoglycerate | 0.047 |
| C50:2 TAG | 0.017 | C22:6 LPE | 0.027 | cAMP | 0.047 |
| C20:3 LPC | 0.017 | putrescine | 0.027 | uridine | 0.047 |
| arginine | 0.017 | C34:1 DAG | 0.027 | PEP | 0.048 |
| C16:0 CE | 0.017 | tryptophan | 0.027 | alpha-glycerophosphate | 0.049 |
| sorbitol | 0.017 | C56:2 TAG | 0.027 | arachidonyl_carnitine | 0.049 |
| C54:4 TAG | 0.017 | uracil | 0.027 | aconitate | 0.050 |
| leucine | 0.018 | histidine | 0.027 | GMP | 0.050 |
| C18:1 CE | 0.018 | C18:0 LPC | 0.027 | adenosine | 0.051 |
| C18:0 SM | 0.018 | C18:1 SM | 0.028 | kynurenic acid | 0.052 |
| C34:4 PC | 0.018 | glutathione reduced | 0.028 | propionylcarnitine | 0.052 |
| C52:3 TAG | 0.018 | C56:3 TAG | 0.028 | glycine | 0.052 |
| pyroglutamic acid | 0.018 | C54:5 TAG | 0.028 | lauroylcarnitine | 0.053 |
| C18:2 SM | 0.018 | AMP | 0.028 | glycodeoxycholate/ | 0.053 |
| | | | | glycochenodeoxycholate | |
| C54:7 TAG | 0.019 | taurodeoxycholate/ | 0.028 | anthranilic acid | 0.053 |
| | | taurochenodeoxycholate | | | |
| C22:6 CE | 0.019 | C14:0 CE | 0.028 | 2-aminoadipate | 0.053 |
| betaine | 0.019 | C18:0 LPE | 0.029 | cystathionine | 0.054 |
| C16:1 CE | 0.019 | C58:7 TAG | 0.029 | thymidine | 0.054 |
| thymine | 0.019 | adipate | 0.029 | thyroxine | 0.055 |
| C20:4 LPC | 0.019 | dimethylglycine | 0.030 | C48:3 TAG | 0.055 |
| creatine | 0.019 | C18:0 CE | 0.030 | glutathione oxidized | 0.057 |
| asparagine | 0.019 | C54:1 TAG | 0.030 | 6-phosphogluconate | 0.058 |
| C16:0 LPC | 0.020 | choline | 0.030 | valerylcarnitine/ | 0.058 |
| | | | | isovalerylcarnitine/ | |
| | | | | 2-methylbutyroylcarnitine | |
| valine | 0.020 | C50:1 TAG | 0.030 | malonylcarnitine | 0.058 |
| lactate | 0.020 | C52:1 TAG | 0.031 | stearoylcarnitine | 0.059 |
| C18:1 LPC | 0.020 | niacinamide | 0.031 | 2-deoxyadenosine | 0.059 |
| C20:4 CE | 0.020 | carnitine | 0.031 | acetylglycine | 0.059 |
| C36:1 DAG | 0.020 | C14:0 LPC | 0.031 | butyrylcarnitine/ | 0.059 |
| | | | | isobutyrylcarnitine | |
| C54:3 TAG | 0.021 | C50:0 TAG | 0.031 | anserine | 0.060 |
| tyrosine | 0.021 | 1-methylnicotinamide | 0.031 | UMP | 0.062 |
| C48:2 TAG | 0.021 | C48:0 TAG | 0.031 | N-carbamoyl-beta-alanine | 0.062 |
| cis/trans-hydroxyproline | 0.021 | trimethylamine-N-oxide | 0.032 | beta-alanine | 0.064 |
| C52:2 TAG | 0.021 | ribose-5-P/ribulose5-P | 0.032 | kynurenine | 0.064 |
| C54:2 TAG | 0.022 | taurine | 0.032 | 5-HIAA | 0.070 |
| C20:5 CE | 0.022 | alanine | 0.033 | ornithine | 0.070 |
| thiamine | 0.022 | 2-hydroxyglutarate | 0.033 | 5-adenosylhomocysteine | 0.071 |
| fumarate/maleate/ | 0.022 | allantoin | 0.033 | hexanoylcarnitine | 0.074 |
| alpha-ketoisovalerate | | | | | |
| C58:6 TAG | 0.022 | C18:1 LPE | 0.033 | heptanoylcarnitine | 0.076 |
| C56:5 TAG | 0.022 | citrulline | 0.034 | cytidine | 0.080 |
| C18:2 CE | 0.022 | NAD | 0.035 | guanosine | 0.081 |
| C16:1 SM | 0.022 | alpha-glycerophosphocholine | 0.035 | NADP | 0.083 |
| alpha-ketoglutarate | 0.022 | inosine | 0.036 | adenine | 0.084 |
| C22:0 SM | 0.023 | CMP | 0.036 | carnosine | 0.084 |
| C52:4 TAG | 0.023 | C16:0 LPE | 0.036 | myristoylcarnitine | 0.086 |
| C56:4 TAG | 0.023 | lysine | 0.036 | palmitoylcarnitine | 0.092 |
| malate | 0.023 | C48:1 TAG | 0.037 | sucrose | 0.096 |
| C14:0 SM | 0.023 | acetylcarnitine | 0.037 | hypoxanthine | 0.097 |
| UDP-galactose/UDP- | 0.023 | 2-deoxycytidine | 0.038 | homocysteine | 0.098 |
| glucose | | | | | |
| C40:6 PC | 0.023 | pipecolic acid | 0.233 | lactose | 0.156 |
| C24:1 SM | 0.023 | acetylcholine | 0.393 | serotonin | 0.207 |

TABLE 3
Lineage effects for each metabolite.

| | Lineage |
|---|---|
| Metabolites | effects |
| phosphocreatine | 0.396 |
| xanthine | 0.365 |
| C20:4 CE | 0.362 |
| 1-methylnicotinamide | 0.339 |
| creatinine | 0.327 |
| kynurenic acid | 0.326 |
| C18:2 CE | 0.320 |
| oxalate | 0.312 |
| lysine | 0.307 |
| C16:1 CE | 0.307 |
| C18:1 CE | 0.305 |
| C16:0 CE | 0.304 |
| C20:5 CE | 0.300 |
| UMP | 0.290 |
| NMMA | 0.289 |
| phenylalanine | 0.286 |
| CMP | 0.282 |
| C38:4 PC | 0.281 |
| leucine | 0.278 |
| C58:6 TAG | 0.278 |
| carnosine | 0.272 |
| hexoses (HILIC neg) | 0.271 |
| tyrosine | 0.271 |
| C38:5 PC | 0.271 |
| methionine | 0.269 |
| AMP | 0.267 |
| C56:5 TAG | 0.264 |
| C56:8 TAG | 0.260 |
| histidine | 0.258 |
| C56:6 TAG | 0.256 |
| C58:8 TAG | 0.255 |
| thiamine | 0.254 |
| dCMP | 0.251 |
| C36:4 PC-B | 0.246 |
| uracil | 0.243 |
| C18:3 CE | 0.241 |
| C40:6 PC | 0.238 |
| pyroglutamic acid | 0.236 |
| arachidonyl_carnitine | 0.234 |
| methionine sulfoxide | 0.232 |
| C56:7 TAG | 0.232 |
| alpha-glycerophosphate | 0.230 |
| cytidine | 0.228 |
| sorbitol | 0.227 |
| SDMA/ADMA | 0.224 |
| C20:3 CE | 0.224 |
| C38:6 PC | 0.222 |
| valine | 0.220 |
| C54:7 TAG | 0.218 |
| C56:4 TAG | 0.218 |
| creatine | 0.217 |
| alpha-hydroxybutyrate | 0.215 |
| isoleucine | 0.215 |
| C54:6 TAG | 0.214 |
| C52:5 TAG | 0.212 |
| C58:7 TAG | 0.209 |
| N-carbamoyl-beta- | 0.208 |
| alanine | |
| allantoin | 0.206 |
| C22:6 CE | 0.201 |
| carnitine | 0.194 |
| thyroxine | 0.193 |
| lactose | 0.193 |
| trimethylamine-N-oxide | 0.192 |
| C54:5 TAG | 0.192 |
| hexoses (HILIC pos) | 0.187 |
| hippurate | 0.183 |
| dimethylglycine | 0.183 |
| tryptophan | 0.180 |
| C46:1 TAG | 0.177 |
| C46:2 TAG | 0.173 |
| threonine | 0.171 |
| C36:4 PC-A | 0.171 |
| DHAP/glyceraldehyde | 0.169 |
| 3P | |
| GMP | 0.169 |
| C54:1 TAG | 0.165 |
| C46:0 TAG | 0.164 |
| myristoylcarnitine | 0.162 |
| glutamate | 0.162 |
| acetylglycine | 0.160 |
| C56:2 TAG | 0.160 |
| anserine | 0.160 |
| guanosine | 0.159 |
| C18:2 SM | 0.157 |
| C22:1 SM | 0.155 |
| C48:2 TAG | 0.153 |
| glutathione oxidized | 0.149 |
| 2-aminoadipate | 0.149 |
| glycodeoxycholate/ | 0.148 |
| glycochenodeoxycholate | |
| C54:4 TAG | 0.148 |
| ribose-5-P/ribulose5-P | 0.148 |
| palmitoylcarnitine | 0.146 |
| cotinine | 0.145 |
| F1P/F6P/G1P/G6P | 0.143 |
| lauroylcarnitine | 0.143 |
| C36:3 PC | 0.143 |
| C18:1 LPC | 0.142 |
| C54:2 TAG | 0.141 |
| C52:4 TAG | 0.141 |
| 3-phosphoglycerate | 0.139 |
| betaine | 0.137 |
| aconitate | 0.136 |
| 3-methyladipate/pimelate | 0.136 |
| xanthosine | 0.135 |
| alanine | 0.134 |
| lactate | 0.133 |
| C36:1 DAG | 0.133 |
| glutathione reduced | 0.133 |
| 6-phosphogluconate | 0.132 |
| C56:3 TAG | 0.130 |
| C48:1 TAG | 0.129 |
| thymidine | 0.128 |
| C32:0 PC | 0.128 |
| NADP | 0.127 |
| C16:0 LPE | 0.127 |
| C50:2 TAG | 0.126 |
| C14:0 LPC | 0.125 |
| 5-adenosylhomocysteine | 0.124 |
| C52:1 TAG | 0.124 |
| C34:2 DAG | 0.123 |
| C50:3 TAG | 0.123 |
| C18:0 CE | 0.122 |
| urate | 0.121 |
| C34:1 PC | 0.120 |
| C52:2 TAG | 0.119 |
| 2-hydroxyglutarate | 0.118 |
| butyrobetaine | 0.118 |
| C20:4 LPE | 0.117 |
| C18:1 LPE | 0.117 |
| arginine | 0.116 |
| citrate | 0.115 |
| 2-deoxycytidine | 0.114 |
| alpha-ketoglutarate | 0.114 |
| succinate/methylmalonate | 0.114 |
| GABA | 0.114 |
| C22:6 LPE | 0.112 |
| C16:0 SM | 0.112 |
| oleylcarnitine | 0.112 |
| C34:1 DAG | 0.112 |
| malonylcarnitine | 0.111 |
| C18:0 SM | 0.109 |
| choline | 0.105 |
| C50:1 TAG | 0.105 |
| C50:0 TAG | 0.105 |
| citrulline | 0.104 |
| C52:3 TAG | 0.103 |
| C16:1 LPC | 0.102 |
| C22:6 LPC | 0.102 |
| C54:3 TAG | 0.101 |
| hypoxanthine | 0.100 |
| acetylcarnitine | 0.100 |
| C16:1 SM | 0.100 |
| anthranilic acid | 0.099 |
| pantothenate | 0.099 |
| beta-alanine | 0.099 |
| C48:3 TAG | 0.097 |
| stearoylcarnitine | 0.097 |
| C18:1 SM | 0.097 |
| C16:0 LPC | 0.097 |
| glycine | 0.096 |
| C36:2 PC | 0.096 |
| taurine | 0.095 |
| C36:2 DAG | 0.095 |
| cystathionine | 0.094 |
| hexanoylcarnitine | 0.094 |
| adenine | 0.093 |
| C22:0 SM | 0.093 |
| taurodeoxycholate/ | 0.093 |
| taurochenodeoxycholate | |
| cis/trans-hydroxyproline | 0.091 |
| inosine | 0.090 |
| pipecolic acid | 0.090 |
| C32:2 PC | 0.089 |
| isocitrate | 0.089 |
| acetylcholine | 0.088 |
| cAMP | 0.086 |
| glucuronate | 0.086 |
| inositol | 0.084 |
| 5-HIAA | 0.084 |
| heptanoylcarnitine | 0.083 |
| C34:4 PC | 0.083 |
| C36:1 PC | 0.083 |
| C24:1 SM | 0.083 |
| C20:4 LPC | 0.082 |
| C48:0 TAG | 0.082 |
| propionylcarnitine | 0.082 |
| adenosine | 0.081 |
| 2-deoxyadenosine | 0.081 |
| sarcosine | 0.081 |
| asparagine | 0.080 |
| 4-pyridoxate | 0.078 |
| C38.2 PC | 0.078 |
| C18:0 LPE | 0.076 |
| niacinamide | 0.074 |
| C20:3 LPC | 0.074 |
| malondialdehyde | 0.074 |
| UDP-galactose/UDP- | 0.072 |
| glucose | |
| putrescine | 0.071 |
| proline | 0.071 |
| glutamine | 0.068 |
| C14:0 CE | 0.068 |
| NAD | 0.068 |
| C24:0 SM | 0.067 |
| butyrylcarnitine/ | 0.067 |
| isobutyrylcarnitine | |
| adipate | 0.066 |
| C34:3 PC | 0.065 |
| C18:0 LPC | 0.063 |
| aspartate | 0.063 |
| C32:1 PC | 0.060 |
| PEP | 0.059 |
| ornithine | 0.058 |
| C34:2 PC | 0.057 |
| serine | 0.055 |
| serotonin | 0.055 |
| C14:0 SM | 0.055 |
| kynurenine | 0.053 |
| homocysteine | 0.052 |
| valerylcarnitine/ | 0.051 |
| isovalerylcarnitine/2- | |
| methylbutyroylcarnitine | |
| alpha- | 0.051 |
| glycerophosphocholine | |
| C18:2 LPC | 0.050 |
| sucrose | 0.050 |
| fumarate/maleate/alpha- | 0.048 |
| ketoisovalerate | |
| taurocholate | 0.047 |
| erythrose-4-phosphate | 0.043 |
| malate | 0.042 |
| thymine | 0.041 |
| uridine | 0.034 |

TABLE 4
Metabolic genes with significant methylation effects on transcripts.

| | | methylation |
|---|---|---|
| Gene | Class | effects |
| AADAT | Amino Acid | 0.669 |
| DDO | Amino Acid | 0.320 |
| ASNS | Amino Acid | 0.134 |
| ACY3 | Amino Acid | 0.295 |
| GPT2 | Amino Acid | 0.271 |
| GLUL | Amino Acid | 0.496 |
| GAD1 | Amino Acid | 0.370 |
| OAT | Amino Acid | 0.455 |
| BHMT2 | Amino Acid | 0.510 |
| AASS | Amino Acid | 0.357 |
| PYCR1 | Amino Acid | 0.488 |
| HGD | Amino Acid | 0.521 |
| FAH | Amino Acid | 0.226 |
| ASL | Amino Acid | 0.253 |
| ASS1 | Amino Acid | 0.576 |
| GNPDA1 | Carbohydrate | 0.567 |
| UAP1L1 | Carbohydrate | 0.461 |
| NANP | Carbohydrate | 0.252 |
| GYG1 | Carbohydrate | 0.138 |
| UGT3A2 | Carbohydrate | 0.184 |
| ENOSF1 | Carbohydrate | 0.580 |
| GALT | Carbohydrate | 0.297 |
| CRYL1 | Carbohydrate | 0.282 |
| GALK1 | Carbohydrate | 0.291 |
| XYLB | Carbohydrate | 0.424 |
| CBS | Glutathione | 0.596 |
| GPX7 | Glutathione | 0.650 |
| GSTM4 | Glutathione | 0.506 |
| GSTM3 | Glutathione | 0.520 |
| MGST3 | Glutathione | 0.435 |
| GPX1 | Glutathione | 0.759 |
| MGST2 | Glutathione | 0.611 |
| GPX3 | Glutathione | 0.559 |
| GSTA4 | Glutathione | 0.311 |
| GSTK1 | Glutathione | 0.321 |
| GSTO1 | Glutathione | 0.491 |
| GSTO2 | Glutathione | 0.676 |
| GSTP1 | Glutathione | 0.688 |
| GPX2 | Glutathione | 0.663 |
| GGT6 | Glutathione | 0.500 |
| GGT7 | Glutathione | 0.508 |
| B4GALT2 | Glycan | 0.507 |
| GTDC1 | Glycan | 0.226 |
| B3GALNT1 | Glycan | 0.714 |
| ST8SIA4 | Glycan | 0.516 |
| B3GALT4 | Glycan | 0.606 |
| FUT9 | Glycan | 0.406 |
| GALNT11 | Glycan | 0.797 |
| GALNT12 | Glycan | 0.431 |
| B4GALNT4 | Glycan | 0.548 |
| B4GALNT1 | Glycan | 0.465 |
| XYLT1 | Glycan | 0.408 |
| ST3GAL2 | Glycan | 0.389 |
| MGAT5B | Glycan | 0.604 |
| B4GALT6 | Glycan | 0.316 |
| B3GNT3 | Glycan | 0.620 |
| MFNG | Glycan | 0.634 |
| A4GALT | Glycan | 0.487 |
| PIGH | Glycan | 0.598 |
| FUCA1 | Glycan | 0.282 |
| MANEAL | Glycan | 0.434 |
| MAN1A2 | Glycan | 0.232 |
| DDUA | Glycan | 0.382 |
| HEXB | Glycan | 0.460 |
| NEU1 | Glycan | 0.462 |
| FUCA2 | Glycan | 0.729 |
| GLB1L2 | Glycan | 0.606 |
| HEXA | Glycan | 0.446 |
| CHST10 | Glycan | 0.591 |
| CHPF | Glycan | 0.462 |
| CHST2 | Glycan | 0.306 |
| CHST3 | Glycan | 0.634 |
| SGSH | Glycan | 0.592 |
| CHST8 | Glycan | 0.406 |
| HPSE | Glycan | 0.360 |
| EXT1 | Glycan | 0.602 |
| HS3ST3B1 | Glycan | 0.321 |
| PGM1 | Glycolysis | 0.378 |
| PFKFB2 | Glycolysis | 0.437 |
| HK2 | Glycolysis | 0.207 |
| PFKP | Glycolysis | 0.332 |
| HK1 | Glycolysis | 0.347 |
| ENO2 | Glycolysis | 0.264 |
| ALDOC | Glycolysis | 0.571 |
| INPP5D | Inositol Phosphate | 0.651 |
| SYNJ2 | Inositol Phosphate | 0.377 |
| PIP4K2A | Inositol Phosphate | 0.357 |
| PI4K2A | Inositol Phosphate | 0.361 |
| INPP5A | Inositol Phosphate | 0.442 |
| PIP4K2C | Inositol Phosphate | 0.615 |
| ISYNA1 | Inositol Phosphate | 0.382 |
| PLCB3 | Inositol Phosphate | 0.481 |
| SUCLG2 | Krebs | 0.436 |
| ME1 | Krebs | 0.718 |
| PC | Krebs | 0.567 |
| ME3 | Krebs | 0.657 |
| AGPS | Lipid | 0.502 |
| ACOT4 | Lipid | 0.574 |
| CRAT | Lipid | 0.722 |
| FAAH | Lipid | 0.466 |
| ECHDC2 | Lipid | 0.593 |
| ACADM | Lipid | 0.368 |
| PECR | Lipid | 0.360 |
| EHHADH | Lipid | 0.589 |
| ELOVL5 | Lipid | 0.635 |
| ELOVL4 | Lipid | 0.563 |
| ACAT2 | Lipid | 0.203 |
| PHYH | Lipid | 0.244 |
| SCD | Lipid | 0.175 |
| ELOVL3 | Lipid | 0.381 |
| FAR1 | Lipid | 0.563 |
| CPT1A | Lipid | 0.322 |
| ACSS3 | Lipid | 0.496 |
| MLYCD | Lipid | 0.530 |
| LIPG | Lipid | 0.500 |
| ECH1 | Lipid | 0.194 |
| MBOAT2 | Lipid | 0.557 |
| PLD1 | Lipid | 0.461 |
| MBOAT1 | Lipid | 0.380 |
| CROT | Lipid | 0.424 |
| DAGLA | Lipid | 0.683 |
| PLA2G16 | Lipid | 0.523 |
| DGKA | Lipid | 0.363 |
| CHPT1 | Lipid | 0.380 |
| DGKE | Lipid | 0.622 |
| AGPAT3 | Lipid | 0.273 |
| PLA2G3 | Lipid | 0.310 |
| THEM4 | Lipid | 0.696 |
| DDHD1 | Lipid | 0.312 |
| ATP10A | Lipid | 0.356 |
| SMPDL3B | Lipid | 0.371 |
| CERK | Lipid | 0.325 |
| HSD17B4 | Lipid | 0.404 |
| HSD17B8 | Lipid | 0.518 |
| HSD17B12 | Lipid | 0.475 |
| ENTPD3 | Nucleotide | 0.448 |
| NME6 | Nucleotide | 0.305 |
| NT5DC2 | Nucleotide | 0.430 |
| NT5DC1 | Nucleotide | 0.345 |
| ENTPD7 | Nucleotide | 0.529 |
| NT5DC3 | Nucleotide | 0.374 |
| NUDT14 | Nucleotide | 0.379 |
| NME4 | Nucleotide | 0.437 |
| NME3 | Nucleotide | 0.586 |
| NTSC | Nucleotide | 0.345 |
| ATP6V0A1 | Nucleotide | 0.555 |
| GDA | Nucleotide | 0.449 |
| DCTD | Nucleotide | 0.241 |
| TK2 | Nucleotide | 0.221 |
| ADCY3 | Nucleotide | 0.155 |
| GUCY1B3 | Nucleotide | 0.445 |
| ADCY1 | Nucleotide | 0.536 |
| PDE3B | Nucleotide | 0.426 |
| GUCY1A2 | Nucleotide | 0.335 |
| ADCY6 | Nucleotide | 0.695 |
| ADCY9 | Nucleotide | 0.514 |
| PDE9A | Nucleotide | 0.383 |
| SMPDL3A | Nucleotide | 0.469 |
| LDHB | Redox | 0.684 |
| SCCPDH | Redox | 0.381 |
| MMACHC | Redox | 0.375 |
| IVD | Redox | 0.644 |
| SPR | Redox | 0.674 |
| QDPR | Redox | 0.393 |
| CYP27A1 | Redox | 0.575 |
| CYP7B1 | Redox | 0.403 |
| DHCR24 | Redox | 0.570 |
| CYP51A1 | Redox | 0.284 |
| SQLE | Redox | 0.241 |
| COX7A2 | Redox | 0.144 |
| COX7A1 | Redox | 0.305 |
| CDO1 | Redox | 0.337 |
| PHYHD1 | Redox | 0.415 |
| ETFA | Redox | 0.254 |
| ETFB | Redox | 0.242 |
| MTHFD2 | Redox | 0.385 |
| ALDH5A1 | Redox | 0.315 |
| PTGR1 | Redox | 0.678 |
| PTGS1 | Redox | 0.425 |
| GPD2 | Redox | 0.617 |
| MSRA | Redox | 0.338 |
| AKR7A3 | Redox | 0.492 |
| ALDH7A1 | Redox | 0.767 |
| AKR1B1 | Redox | 0.677 |
| ALDH1B1 | Redox | 0.330 |
| ALDH3B1 | Redox | 0.652 |
| ALDH1L2 | Redox | 0.480 |
| ALDH2 | Redox | 0.576 |
| ALDH3A2 | Redox | 0.443 |
| ALDH3A1 | Redox | 0.495 |
| ALDH16A1 | Redox | 0.349 |
| CBR1 | Redox | 0.764 |
| CBR3 | Redox | 0.532 |
| NNT | Redox | 0.576 |
| NQO1 | Redox | 0.646 |
| CHDH | Redox | 0.460 |
| WWOX | Redox | 0.271 |
| PAOX | Redox | 0.339 |
| SMOX | Redox | 0.315 |
| BLVRA | Redox | 0.489 |
| ALDH4A1 | Redox | 0.370 |
| PRDX1 | Redox | 0.231 |
| CYBRD1 | Redox | 0.460 |
| TXNRD3 | Redox | 0.646 |
| CYBA | Redox | 0.750 |
| CYB561 | Redox | 0.661 |
| CYB5A | Redox | 0.532 |
| PRDX2 | Redox | 0.649 |
| TXNRD2 | Redox | 0.203 |
| PHGDH | Redox | 0.355 |
| CYP2R1 | Redox | 0.405 |
| CYP2S1 | Redox | 0.599 |
| HSD17B14 | Redox | 0.355 |
| SRXN1 | Redox | 0.600 |
| HPDL | Redox | 0.661 |
| CYP26C1 | Redox | 0.187 |
| ABCA1 | Transport | 0.361 |
| ABCC4 | Transport | 0.279 |
| ABCA3 | Transport | 0.321 |
| ABCC3 | Transport | 0.668 |
| ABCG1 | Transport | 0.406 |
| SLC25A33 | Transport | 0.444 |
| SLC6A17 | Transport | 0.501 |
| SLC16A1 | Transport | 0.410 |
| SLC19A2 | Transport | 0.456 |
| SLC4A3 | Transport | 0.638 |
| SLC16A14 | Transport | 0.503 |
| SLC4A7 | Transport | 0.235 |
| SLC25A38 | Transport | 0.421 |
| SLC25A20 | Transport | 0.550 |
| SLC7A14 | Transport | 0.369 |
| SLC2A9 | Transport | 0.347 |
| SLC25A4 | Transport | 0.356 |
| SLCO4C1 | Transport | 0.381 |
| SLC25A46 | Transport | 0.564 |
| SLC44A4 | Transport | 0.563 |
| SLC29A4 | Transport | 0.453 |
| SLC25A13 | Transport | 0.384 |
| SLC37A3 | Transport | 0.521 |
| SLC35D2 | Transport | 0.781 |
| SLC2A8 | Transport | 0.676 |
| SLC2A6 | Transport | 0.423 |
| SLC43A3 | Transport | 0.672 |
| SLC29A2 | Transport | 0.466 |
| SLC36A4 | Transport | 0.505 |
| SLC35F2 | Transport | 0.324 |
| SLC38A1 | Transport | 0.435 |
| SLC6A15 | Transport | 0.567 |
| SLC15A4 | Transport | 0.322 |
| SLC46A3 | Transport | 0.402 |
| SLC25A30 | Transport | 0.342 |
| SLC22A17 | Transport | 0.477 |
| SLC25A21 | Transport | 0.435 |
| SLC25A29 | Transport | 0.308 |
| SLCO3A1 | Transport | 0.395 |
| SLC7A5 | Transport | 0.336 |
| SLC16A13 | Transport | 0.433 |
| SLC47A1 | Transport | 0.377 |
| SLC46A1 | Transport | 0.469 |
| SLC16A5 | Transport | 0.649 |
| SLC2A10 | Transport | 0.520 |
| SLC7A4 | Transport | 0.313 |
| CKB | Other | 0.423 |
| THNSL2 | Other | 0.602 |
| PCBD1 | Other | 0.642 |
| MOCS1 | Other | 0.568 |
| GPHN | Other | 0.607 |
| MOCOS | Other | 0.670 |
| GAMT | Other | 0.620 |
| EPHX2 | Other | 0.478 |
| ECHDC1 | Other | 0.560 |
| ECHDC3 | Other | 0.652 |
| HS6ST1 | Other | 0.326 |
| HS3ST1 | Other | 0.413 |
| DIO3 | Other | 0.317 |
| ACE | Other | 0.479 |
| OXCT1 | Other | 0.448 |
| PLCL1 | Other | 0.195 |
| GK5 | Other | 0.332 |
| ABHD1 | Other | 0.194 |
| ABHD10 | Other | 0.280 |
| NAT8L | Other | 0.431 |
| HMGCLL1 | Other | 0.448 |
| GGH | Other | 0.449 |
| CA2 | Other | 0.312 |
| ABHD8 | Other | 0.437 |
| QPRT | Other | 0.396 |
| NUDT7 | Other | 0.320 |
| NUDT19 | Other | 0.527 |
| IAH1 | Other | 0.668 |
| PON2 | Other | 0.682 |
| PTER | Other | 0.619 |
| ESD | Other | 0.235 |
| PCCA | Other | 0.349 |
| UCP2 | Other | 0.577 |
| SGPP1 | Other | 0.314 |
| GALC | Other | 0.644 |
| SULT2B1 | Other | 0.461 |
| MPST | Other | 0.389 |
| SULT4A1 | Other | 0.505 |
| AGMAT | Other | 0.629 |
| LRAT | Other | 0.465 |
| OGDHL | Other | 0.400 |

### REFERENCES

1. Vander Heiden, M. G. et al. Understanding the Warburg effect: the metabolic requirements of cell proliferation. Science 324, 1029-1034 (2009).
2. Pavlova, N. N. & Thompson, C. B. The emerging hallmarks of cancer metabolism. Cell Metabolism 23, 27-47 (2016).
3. Cairns, R. A., Harris, I. S. & Mak, T. W. Regulation of cancer cell metabolism. Nature Reviews Cancer 11, 85-95 (2011).
4. Vander Heiden, M. G. & Deberardinis, R. J. Understanding the intersections between metabolism and cancer biology. Cell 168, 657-669 (2017).
5. Tennant, D. A., Duran, R. V & Gottlieb, E. Targeting metabolic transformation for cancer therapy. Nature Reviews Cancer 10, 267-277 (2010).
6. McDonald, E. R. I. et al. Project DRIVE: a compendium of cancer dependencies and synthetic lethal relationships uncovered by large-Scale, deep RNAi screening. Cell 170, 577-592 (2017).
7. Barretina, J. et al. The Cancer Cell Line Encyclopedia enables predictive modelling of anticancer drug sensitivity. Nature 483, 603-7 (2012).
8. Tsherniak, A. et al. Defining a cancer dependency map. Cell 170, 564-576 (2017).
9. Xu, W. et al. Oncometabolite 2-hydroxyglutarate is a competitive inhibitor of a ketoglutarate-dependent dioxygenases. Cancer Cell 19, 17-30 (2011).
10. Dang, L. et al. Cancer-associated IDH1 mutations produce 2-hydroxyglutarate. Nature 462, 739-744 (2009).
11. Shim, E. H. et al. L-2-hydroxyglutarate: An epigenetic modifier and putative oncometabolite in renal cancer. Cancer Discovery 4, 1290-1298 (2014).
12. Wakabayashi, N. et al. Protection against electrophile and oxidant stress by induction of the phase 2 response: Fate of cysteines of the Keap1 sensor modified by inducers. Proceedings of the National Academy of Sciences of the United States of America 101, 2040-2045 (2004).
13. Ohta, T. et al. Loss of Keap1 function activates Nrf2 and provides advantages for lung cancer cell growth. Cancer Research 68, 1303-1309 (2008).
14. Dey, P. et al. Genomic deletion of malic enzyme 2 confers collateral lethality in pancreatic cancer. Nature 542, 119-123 (2017).
15. Meyers, R. M. et al. Computational correction of copy number effect improves specificity of CRISPR-Cas9 essentiality screens in cancer cells. Nature genetics (2017). doi:10.1038/ng.3984
16. Indiveri, C., Tonazzi, A., Prezioso, G. & Palmieri, F. Kinetic characterization of the reconstituted carnitine carrier from rat liver mitochondria. Biochimica et Biophysica Acta 1065, 231-238 (1991).
17. Yang, W. S. et al. Regulation of ferroptotic cancer cell death by GPX4. Cell 156, 317-331 (2014).
18. Yu, C. et al. High-throughput identification of genotype-specific cancer vulnerabilities in mixtures of barcoded tumor cell lines. Nature biotechnology 34, 419-423 (2016).
19. Mezrich, J. D. et al. An Interaction between kynurenine and the aryl hydrocarbon receptor can generate regulatory T cells. The Journal of Immunology 185, 3190-3198 (2010).
20. Uyttenhove, C. et al. Evidence for a tumoral immune resistance mechanism based on tryptophan degradation by indoleamine 2,3-dioxygenase. Nature Medicine 9, 1269-1274 (2003).
21. Munn, D. H. & Mellor, A. L. Indoleamine 2,3 dioxygenase and metabolic control of immune responses. Trends in Immunology 34, 137-143 (2013).
22. Brochez, L., Chevolet, I. & Kruse, V. The rationale of indoleamine 2, 3-dioxygenase inhibition for cancer therapy. European Journal of Cancer 76, 167-182 (2017).
23. Jain, M. et al. Metabolite profiling identifies a key role for glycine in rapid cancer cell proliferation. Science 336, 1040-1044 (2012).
24. Long, G. V et al. Epacadostat (E) plus pembrolizumab (P) versus pembrolizumab alone in patients (pts) with unresectable or metastatic melanoma: results of the phase 3 ECHO-301/KEYNOTE-252 study. in J Clin Oncol 36, 2018 (suppl; abstr 108) (2018).
25. Narta, U. K., Kanwar, S. S. & Azmi, W. Pharmacological and clinical evaluation of L-asparaginase in the treatment of leukemia. Critical Reviews in Oncology/Hematology 61, 208-221(2007).
26. Hettmer, S. et al. Functional genomic screening reveals asparagine dependence as a metabolic vulnerability in sarcoma. eLife 4, 1-17 (2015).
27. Knott, S. R. V. et al. Asparagine bioavailability governs metastasis in a model of breast cancer. Nature 554, 378-381 (2018).
28. Townsend, M. K. et al. Reproducibility of metabolomic profiles among men and women in 2 large cohort studies. Clinical Chemistry 59, 1657-1667 (2013).

1. A method for treating liver cancer or stomach cancer in a subject, the method comprising:
(a) detecting a level of asparaginase (ASNS) in a biological sample from a subject, and
(b) administering an effective amount of a pharmaceutical composition comprising ASNS to the subject if the biological sample from the subject exhibits a decreased level of ASNS compared to the level of ASNS in a control sample or compared to a predetermined reference level of ASNS.

2. The method of claim 1, wherein step (a) comprises detecting a level of ASNS protein.

3. The method of claim 2, wherein the level of ASNS protein is detected by an immunohistochemical assay, an immunoblotting assay, or a flow cytometry assay.

4. The method of claim 1, wherein step (a) comprises detecting a level of a nucleic acid encoding ASNS.

5. The method of claim 4, wherein the level of a nucleic acid encoding ASNS is detected by a real-time reverse transcriptase polymerase chain reaction (RT-PCR) assay or a nucleic acid microarray assay.

6. The method of claim 1, wherein step (a) comprises detecting a level of methylation of a ASNS promotor sequence.

7. The method of claim 6, wherein the level of methylation is detected using a hybridization assay, a sequencing assay, or a polymerase chain reaction (PCR) assay.

8. The method of any one of claims 1-7, wherein the biological sample is a tissue sample or a blood sample.

9. The method of any one of claims 1-8, wherein the subject is a human patient having, suspected of having, or at risk for having, liver cancer or stomach cancer.

10. The method of any one of claims 1-9, wherein the control sample is obtained from a human patient that is undiagnosed with cancer.

11. The method of any one of claims 1-9, wherein the predetermined reference level is a level of ASNS from a human patient that is undiagnosed with cancer.

12. The method of any one of claims 1-11, wherein step (b) comprises administering ASNS intravenously or intramuscularly.

13. The method of any one of claims 1-12, further comprising administering to the subject an additional anti-cancer agent.

14. A method for treating liver cancer or stomach cancer in a subject, the method comprising administering to a subject in need thereof an effective amount of a pharmaceutical composition comprising asparaginase (ASNS).

15. The method of claim 14, wherein the subject is a human patient having, suspected of having, or at risk for having liver cancer or stomach cancer.

16. The method of claim 14 or 15, further comprising administering to the subject an additional anti-cancer agent.

17. The method of any one of claims 14-16, wherein the pharmaceutical composition is administered to the subject intravenously or intramuscularly.

18. The method of any one of claims 14-17, wherein the pharmaceutical composition comprises ASNS from Erwinia chrysanthemi.
